(19) United States
(12) Patent Application Publication
(10) Pub. No.: US 2026/0007625 A1
(43) Pub. Date: Jan. 8, 2026

(54) Compounds for the Treatment of Neuromuscular Disorders

(71) Applicant: NMD PHARMA A/S, Aarhus N (DK)

(72) Inventors: Thomas Holm PEDERSEN, Aarhus N (DK); Lars J.S. KNUTSEN, Aarhus N (DK); Nicholas Michael KELLY, Aarhus N (DK); Farman ULLAH, Aarhus N (DK); Neerja SARASWAT, Aarhus N (DK); Rajesh KUMAR, Aarhus N (DK)

(73) Assignee: NMD PHARMA A/S, Aarhus N (DK)

(21) Appl. No.: 19/111,725

(22) PCT Filed: Sep. 15, 2023
(86) PCT No.: PCT/EP2023/075444

§ 371 Date: Mar. 13, 2025

(30) Foreign Application Priority Data
Sep. 15, 2022 (EP) 22195876.2

(51) Int. Cl. A61K 31/192; A61P 21/00; C07C 65/21; C07C 323/53; C07C 323/62

(52) U.S. Cl. CPC A61K 31/192; A61P 21/00; C07C 65/21; C07C 323/53; C07C 323/62

(57) ABSTRACT

The present disclosure relates to compounds suitable for treating, ameliorating and/or preventing neuromuscular disorders, including the reversal of drug-induced neuromuscular blockade. The compounds as defined herein can inhibit the CIC-1 ion channel.

### CROSS-REFERENCE TO RELATED APPLICATION

This application claims priority to International Application No. PCT/EP2023/075444 filed on Sep. 15, 2023, which claims priority to EP Application Serial No. 22195876.2, filed on Sep. 15, 2022, the entire contents of which are incorporated herein by reference in their entireties for all purposes.

### TECHNICAL FIELD

The present disclosure relates to compounds and their use in treating, ameliorating and/or preventing neuromuscular disorders, including the reversal of drug-induced neuromuscular blockade. The compounds as defined herein can inhibit the CIC-1 ion channel. The disclosure further relates to methods of treating, preventing and/or ameliorating neuromuscular disorders, by administering said composition to a person in need thereof.

### BACKGROUND

Walking, breathing, and eye movement are examples of essential everyday physiological activities that are powered by the contractile activity of skeletal muscle. Skeletal muscles are inherently in a resting state and contractile activity occurs exclusively in response to commands from the central nervous system (CNS). Such neuronal commands take the form of action potentials that travel from the brain to the muscle fibres in several steps. The neuromuscular junction (NMJ) is a highly specialized membrane area on muscle fibres where motor neurons come into close contact with the muscle fibres, and it is at the NMJ where neuronal action potentials are transmitted to muscular action potentials in a one-to-one fashion via synaptic transmission.

Unfortunately, none of the currently employed drug regimens for treating neuromuscular disorders, such as myasthenia gravis, Lambert-Eaton Syndrome, Charcot-Marie Tooth disease, amyotrophic lateral sclerosis (ALS), spinal muscular atrophy (SMA) and sarcopenia, have been able to fully restore muscle function in compromised patients.

The CIC-1 ion channel (Pedersen, T. H., Riisager, A., Vincenzo de Paoli, F., Chen, T-Y, Nielsen, O. B. Role of physiological CIC-1 Cl⁻ ion channel regulation for the excitability and function of working skeletal muscle. J. Gen. Physiol. 2016, 147, 291-308) is emerging as a target for improving muscle function in patients having a neuromuscular disfunction.

Kobrina et al (J. Org. Chem. 1970, 6 (1), 510-517) discloses nucleophilic aromatic substitution of some substituted pentafluorobeneze derivatives, including the compound ethyl 2-ethoxy-3,4,5,6-tetrafluorobenzoate. However, the compound is different to the current invention as R² may only be F when R¹ is optionally substituted —OC₂₋₃ alkyl.

EP3 753 560 and WO 2020/254559 both describe the use of CIC-1 inhibitors in the treatment of neuromuscular diseases but the compounds disclosed therein are structurally different to the present invention.

### SUMMARY

The present disclosure is directed to novel CIC-1 ion channel inhibitors for use in the treatment of a range of conditions, such as neuromuscular diseases and reversal of neuromuscular blockade after surgery, in which muscle activation by the nervous system is compromised and symptoms of weakness and fatigue are prominent.

In one aspect, the disclosure concerns a compound of Formula (I):

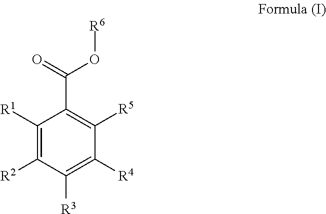

wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸,
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H, Me or Et; and with the proviso that when R¹ is Cl or Br, R² is Me, R³ is H, R⁴ is F and R⁵ is OMe, then R⁶ is not H, Me or Et; and with the proviso that when R¹ is F, R² is Me, R³ is F, R⁴ is F and R⁵ is OMe, then R⁶ is not H.

In another aspect, the disclosure concerns a compound as defined herein for use in treating, ameliorating and/or preventing a neuromuscular disorder, and/or for use in reversing and/or ameliorating a neuromuscular blockade. In yet another aspect, the disclosure concerns a composition comprising a compound as defined herein.

### Definitions

The terms “C₁₋₃ alkyl”, “C₂₋₃ alkyl” and “C₁₋₅ alkyl” refers to a branched or unbranched alkyl group having from one to three, two to three or one to five carbon atoms respectively, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl and pent-3-yl.

The terms “C₂₋₃ alkenyl” and “C₂₋₅ alkenyl” refers to a branched or unbranched alkenyl group having from two to three or two to five carbon atoms respectively, two of which are connected by a double bond, including but not limited to ethenyl, prop-1-enyl, prop-2-enyl, but-1-enyl, but-2-enyl, but-3-enyl, buta-1,3-dienyl, pent-1-enyl, pent-2-enyl, pent-3-enyl, pent-4-enyl, penta-2,4-dienyl and penta-1,3-dienyl.

The term “C₂₋₅ alkynyl” refers to a branched or unbranched alkynyl group having from two to five carbon atoms, two of which are connected by a triple bond, including but not limited to ethynyl, prop-1-ynyl, prop-2-ynyl, but-1-ynyl, but-2-ynyl, but-3-ynyl, buta-1,3-diynyl, pent-1-ynyl, pent-2-ynyl, pent-3-ynyl, pent-4-ynyl, penta-2,4-diynyl and penta-1,3-diynyl.

The term “C₃₋₅ cycloalkyl” and “C₃₋₆ cycloalkyl” refers to a group having three to five or three to six carbon atoms respectively including a monocyclic or bicyclic carbocycle, including but not limited to cyclopropyl, cyclobutyl, cyclopropylmethyl, cyclopentyl, cyclobutylmethyl, cyclopropylethyl, and cyclohexyl.

The term “half-life” as used herein is the time it takes for the compound to lose one-half of its pharmacologic activity. The term “plasma half-life” is the time that it takes the compound to lose one-half of its pharmacologic activity in the blood plasma.

The term “treatment” refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof. In some embodiments, the term “treatment” encompasses amelioration and prevention.

The term “amelioration” refers to moderation in the severity of the symptoms of a disease or condition. Improvement in a patient's condition, or the activity of making an effort to correct, or at least make more acceptable, conditions that are difficult to endure related to patient's conditions is considered “ameliorative” treatment.

The term “prevent” or “preventing” refers to precluding, averting, obviating, forestalling, stopping, or hindering something from happening, especially by advance action.

The term “reversal” or “reversing” refers to the ability of a compound to restore nerve-stimulated force in skeletal muscle exposed either ex vivo or in vivo to a non-depolarizing neuromuscular blocking agent or another pharmaceutical that is able to depress neuromuscular transmission

The term “non-depolarizing blockers” refers to pharmaceutical agents that antagonize the activation of acetylcholine receptors at the post-synaptic muscle fibre membrane by blocking the acetylcholine binding site on the receptor. These agents are used to block neuromuscular transmission and induce muscle paralysis in connection with surgery.

The term “ester hydrolysing reagent” refers to a chemical reagent which is capable of converting an ester functional group to a carboxylic acid with elimination of the alcohol moiety of the original ester, including but not limited to acid, base, a fluoride source, PBr₃, PCl₃ and lipase enzymes.

The term “total membrane conductance (Gm)” is the electrophysiological measure of the ability of ions to cross the muscle fibre surface membrane. It reflects the function of ion channels that are active in resting muscle fibres of which CIC-1 is known to contribute around 80% in most animal species.

### DETAILED DESCRIPTION

### Compounds

It is within the scope of the present disclosure to provide a compound for use in treating, ameliorating and/or preventing neuromuscular disorders that reduce neuromuscular function. As disclosed herein, inhibition of CIC-1 improves or restores neuromuscular function. The compounds of the present disclosure comprise compounds capable of inhibiting the CIC-1 channel thereby improving or restoring neuromuscular function.

In one aspect, the disclosure concerns a compound of Formula (I):

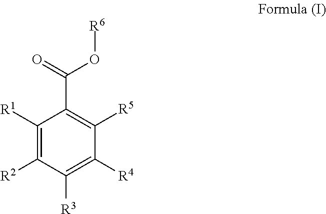

wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H, Me or Et; and with the proviso that when R¹ is Cl or Br, R² is Me, R³ is H, R⁴ is F and R⁵ is OMe, then R⁶ is not H, Me or Et; and with the proviso that when R¹ is F, R² is Me, R³ is F, R⁴ is F and R⁵ is OMe, then R⁶ is not H.

In one embodiment, R¹ is F. In one embodiment, R¹ is C₁. In one embodiment, R¹ is Br. In one embodiment, R¹ is I. In one embodiment, R¹ is C₁₋₃ alkyl optionally substituted with one or more, identical or different, substituents R⁸. In one embodiment, R¹ is —OC₂₋₃ alkyl optionally substituted with one or more, identical or different, substituents R⁷. In one embodiment, R¹ is —OC₃₋₅ cycloalkyl optionally substituted with one or more, identical or different, substituents R⁷. In one embodiment, R¹ is —SC₁₋₃ alkyl optionally substituted with one or more, identical or different, substituents R⁷. In one embodiment, R¹ is —SC₃₋₅ cycloalkyl optionally substituted with one or more, identical or different, substituents R⁷.

In one embodiment, R¹ is selected from the group consisting of F, Cl, Br, and C₁₋₃ alkyl optionally substituted with one or more, identical or different, substituents R⁸. In one embodiment, R¹ is selected from the group consisting of Et, Me or Cl. In one embodiment, R¹ is Me.

In one embodiment, R² is C₁₋₃ alkyl optionally substituted with one or more, identical or different, substituents R⁸. In one embodiment, R² is Me, Et or Pr. In one embodiment, R² is Me. In one embodiment, R² is C₂₋₃ alkenyl optionally substituted with one or more, identical or different, substituents R⁸. In one embodiment, R² is F. In one embodiment, R² is Cl. In one embodiment, R² is Br. In one embodiment, R² is I.

In one embodiment, R³ is H. In one embodiment, R³ is F.

In one embodiment, R⁴ is selected from the group consisting of F, Cl and Br. In one embodiment, R⁴ is selected from the group consisting of F and Cl. In one embodiment, R⁴ is Cl. In one embodiment, R⁴ is F. In one embodiment, R⁴ is Br.

In one embodiment, R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷ and —SC₁₋₃ alkyl optionally substituted with one or more, identical or different, substituents R⁷. In one embodiment, R⁵ is selected from the group consisting of —OC₁₋₃ alkyl optionally substituted with one or more, identical or different, substituents R⁷ and —OC₃₋₅ cycloalkyl optionally substituted with one or more, identical or different, substituents R⁷.

In one embodiment, R⁵ is selected from the group consisting of —OC₁₋₃ alkyl optionally substituted with one or more, identical or different, substituents R⁷ and —SC₁₋₃ alkyl optionally substituted with one or more, identical or different, substituents R⁷. In one embodiment, R⁵ is selected from the group consisting of OC₁₋₃ alkyl optionally substituted with one or more, identical or different, substituents R⁷. In one embodiment, R⁵ is selected from the group consisting of —SC₁₋₃ alkyl optionally substituted with one or more, identical or different, substituents R⁷. In one embodiment, R⁵ is selected from the group consisting of —OC₃₋₅ cycloalkyl optionally substituted with one or more, identical or different, substituents R⁷. In one embodiment, R⁵ is selected from the group consisting of —SC₃₋₅ cycloalkyl optionally substituted with one or more, identical or different, substituents R⁷. In one embodiment, R⁵ is selected from the group consisting of cyclopropoxy, —OEt, —OMe, —OCH₂F, —OCHF₂, —OCF₃, —SMe, —SCH₂F, —SCHF₂ and —SCF₃. In one embodiment, R⁵ is selected from the group consisting of —OMe, —OCH₂F, —OCHF₂, —OCF₃ and —SMe.

In one embodiment, R⁶ is H.

In one embodiment, R⁷ is D. In one embodiment, R⁷ is F. In one embodiment, R⁷ is Cl.

In one embodiment, R⁸ is D. In one embodiment, R⁸ is F. In one embodiment, R⁸ is Cl.

In one embodiment, R⁸ is SMe. In one embodiment, R⁸ is OMe.

In one embodiment, the EC₅₀ of the compound is <10 μM, such as <5 μM, such as <2 μM, and such as <1 μM.

In one aspect, the disclosure concerns a compound of Formula (I):

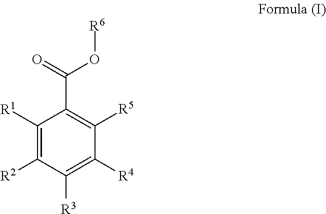

wherein:
   R¹ is selected from the group consisting of F; Cl; Br and C₁₋₃ alkyl optionally substituted with one or more, identical or different, substituents R⁸;
   R² is selected from the group consisting of C₁₋₃ alkyl optionally substituted with one or more, identical or different, substituents R⁸;
   R³ is H;
   R⁴ is selected from the group consisting of F, Cl and Br;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; and —SC₁₋₃ alkyl optionally substituted with one or more, identical or different, substituents R⁷;
   R⁶ is H;
   R⁷ is F; and
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl;
or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H; and with the proviso that when R¹ is Cl or Br, R² is Me, R³ is H, R⁴ is F and R⁵ is OMe, then R⁶ is not H.

In one aspect, the compound is selected from the group consisting of:
3-chloro-2-cyclopropoxy-5,6-dimethylbenzoic acid;
2,5-dichloro-6-methoxy-3-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid; and
3-chloro-2-ethoxy-5,6-dimethylbenzoic acid.

In one embodiment, the compound or the compound for use according to the present disclosure has been modified in order to increase its half-life when administered to a patient, in particular its plasma half-life.

In one embodiment, the compound or the compound for use according to the present disclosure further comprises a moiety conjugated to said compound, thus generating a moiety-conjugated compound. In one embodiment, said moiety-conjugated compound has a plasma and/or serum half-life being longer than the plasma and/or serum half-life of the non-moiety conjugated compound.

In one embodiment, the moiety conjugated to the compound or compound for use according to the present disclosure, is one or more type(s) of moieties selected from the group consisting of albumin, fatty acids, polyethylene glycol (PEG), acylation groups, antibodies and antibody fragments.

### Neuromuscular Disorders

The compound or compound for use of the present disclosure may be used for treating, ameliorating and/or preventing a neuromuscular disorder, or reversing neuromuscular blockade.

The inventors of the present disclosure have shown that inhibition of CIC-1 channels strengthens neuromuscular transmission. CIC-1 function may therefore contribute to muscle weakness in conditions of compromised neuromuscular transmission.

Thus, in one embodiment of the present disclosure, the compound or the compound for use as described herein inhibits CIC-1 channels. Thus, it is appreciated that compounds and/or compounds for use of Formula (I) inhibit CIC-1 channels.

The neuromuscular disorder may also include neuromuscular dysfunctions.

Neuromuscular disorders include for example disorders with symptoms of muscle weakness and fatigue. Such disorders may include conditions with reduced neuromuscular transmission safety factor. In one embodiment the neuromuscular disorders are motor neuron disorders. Motor neuron disorders are disorders with reduced safety in the neuromuscular transmission. In one embodiment motor neuron disorders are selected from the group consisting of amyotrophic lateral sclerosis (ALS) (Killian J M, Wilfong A A, Burnett L, Appel S H, Boland D. Decremental motor responses to repetitive nerve stimulation in ALS. Muscle Nerve, 1994, 17, 747-754), spinal muscular atrophy (SMA) (Wadman R I, Vrancken A F, van den Berg L H, van der Pol W L. Dysfunction of the neuromuscular junction in spinal muscular atrophy types 2 and 3. Neurology, 2012, 79, 2050-2055), Charcot-Marie Tooth disease (Bansagi B, Griffin H, Whittaker R G, Antoniadi T, Evangelista T, Miller J, Greenslade M, Forester N, Duff J, Bradshaw A, Kleinle S, Boczonadi V, Steele H, Ramesh V, Franko E, Pyle A, Lochmüller H, Chinnery P F, Horvath R. Genetic heterogeneity of motor neuropathies. Neurology, 2017, 28; 88 (13): 1226-1234), X-linked spinal and bulbar muscular atrophy (Yamada, M., Inaba, A., Shiojiri, T. X-linked spinal and bulbar muscular atrophy with myasthenic symptoms. Journal of the Neurological Sciences, 1997, 146, 183-185), Kennedy's disorder (Stevic, Z., Peric, S., Pavlovic, S., Basta, I., Lavrnic, D., Myasthenic symptoms in a patient with Kennedy's disorder. Acta Neurologica Belgica, 2014, 114, 71-73), multifocal motor neuropathy (Roberts, M., Willison, H. J., Vincent, A., Newsom-Davis, J. Multifocal motor neuropathy human sera block distal motor nerve conduction in mice. Ann Neurol. 1995, 38, 111-118), Guillain-Barré syndrome (Ansar, V., Valadi, N. Guillain-Barré Syndrome Prim. Care, 2015, 42, 189-193); poliomyelitis (Trojan, D. A., Gendron, D., Cashman, N. R. Electrophysiology and electrodiagnosis of the post-polio motor unit. Orthopedics, 1991, 14, 1353-1361, and Birk T. J. Poliomyelitis and the post-polio syndrome: exercise capacities and adaptation—current research, future directions, and widespread applicability. Med. Sci. Sports Exerc., 1993, 25, 466-472), post-polio syndrome (Garcia, C. C., Potian, J. G., Hognason, K., Thyagarajan, B., Sultatos, L. G., Souayah, N., Routh, V. H., McArdle, J. J. Acetylcholinesterase deficiency contributes to neuromuscular junction dysfunction in type 1 diabetic neuropathy. Am. J. Physiol. Endocrinol. Metab., 2012, 15, E551-561) and sarcopenia (Gilmore K. J., Morat T., Doherty T. J., Rice C. L., Motor unit number estimation and neuromuscular fidelity in 3 stages of sarcopenia. 2017 55(5): 676-684). In one embodiment, the neuromuscular disorder is diabetic polyneuropathy. In one embodiment, the neuromuscular disorder is sarcopenia. In one embodiment, the neuromuscular disorder is Kennedy's disorder. In one embodiment, the neuromuscular disorder is multifocal motor neuropathy.

Thus, in one embodiment of the present disclosure the neuromuscular disorder is amyotrophic lateral sclerosis (ALS). In another embodiment the neuromuscular disorder is spinal muscular atrophy (SMA). In another embodiment the neuromuscular disorder is Charcot-Marie tooth disease (CMT). In another embodiment the neuromuscular disorder is sarcopenia. In yet another embodiment, the neuromuscular disorder is critical illness myopathy (CIM).

As stated above the neuromuscular disorders include for example disorders with symptoms of muscle weakness and fatigue. Such disorder may for example include diabetes (Am. J. Physiol. Endocrinol. Metab., 2012, 15, E551-561).

In another embodiment the neuromuscular disorders is chronic fatigue syndrome. Chronic fatigue syndrome (CFS) (Fletcher, S. N., Kennedy, D. D., Ghosh, I. R., Misra, V. P., Kiff, K., Coakley, J. H., Hinds, C. J. Persistent neuromuscular and neurophysiologic abnormalities in long-term survivors of prolonged critical illness. Crit. Care Med. 2003, 31, 1012-1016) is the common name for a medical condition characterized by debilitating symptoms, including fatigue that lasts for a minimum of six months in adults. CFS may also be referred to as systemic exertion intolerance disorder (SEID), myalgic encephalomyelitis (ME), post-viral fatigue syndrome (PVFS), chronic fatigue immune dysfunction syndrome (CFIDS), or by several other terms. Symptoms of CFS include malaise after exertion; unrefreshing sleep, widespread muscle and joint pain, physical exhaustion, and muscle weakness.

In a further embodiment the neuromuscular disorder is a critical illness polyneuropathy (Angelini C. Spectrum of metabolic myopathies. Biochim. Biophys. Acta., 2015, 1852, 615-621) or CIM (Latronico, N., Bolton, C. F. Critical illness polyneuropathy and myopathy: a major cause of muscle weakness and paralysis. Lancet Neurol. 2011, 10, 931-941). Critical illness polyneuropathy and CIM are overlapping syndromes of widespread muscle weakness and neurological dysfunction developing in critically ill patients.

The neuromuscular disorder may also include metabolic myopathy (Milone, M., Wong, L. J. Diagnosis of mitochondrial myopathies. Mol. Genet. Metab., 2013, 110, 35-41) and mitochondrial myopathy (Srivastava, A., Hunter, J. M. Reversal of neuromuscular block. Br. J. Anaesth. 2009, 103, 115-129). Metabolic myopathies result from defects in biochemical metabolism that primarily affects muscle. These may include glycogen storage disorders, lipid storage disorder and 3-phosphocreatine stores disorder. Mitochondrial myopathy is a type of myopathy associated with mitochondrial disorder. Symptoms of mitochondrial myopathies include muscular and neurological problems such as muscle weakness, exercise intolerance, hearing loss and trouble with balance and coordination. Thus, in one embodiment of the present disclosure the neuromuscular disorder is metabolic myopathy.

In another embodiment the neuromuscular disorder is periodic paralysis, in particular hypokalemic periodic paralysis which is a disorder of skeletal muscle excitability that presents with recurrent episodes of weakness, often triggered by exercise, stress, or carbohydrate-rich meals (Wu, F., Mi, W., Cannon, S. C., Neurology, 2013, 80, 1110-1116 and Suetterlin, K. et at, Current Opinion Neurology, 2014, 27, 583-590) or hyperkalemic periodic paralysis which is an inherited autosomal dominant disorder that affects sodium channels in muscle cells and the ability to regulate potassium levels in the blood (Ammat, T. et al, Journal of General Physiology, 2015, 146, 509-525).

In an embodiment the neuromuscular disorder is a myasthenic condition. Myasthenic conditions are characterized by muscle weakness and neuromuscular transmission failure. Congenital myasthenic syndrome (Finlayson, S., Beeson, D., Palace, J. Congenital myasthenic syndromes: an update. Pract. Neurol., 2013, 13, 80-91) is an inherited neuromuscular disorder caused by defects of several types at the neuromuscular junction.

Myasthenia gravis and Lambert-Eaton syndrome (Titulaer M. J., Lang B., Verschuuren J J. Lambert-Eaton myasthenic syndrome: from clinical characteristics to therapeutic strategies. Lancet Neurol. 2011, 10, 1098-107) are examples of myasthenic conditions. Myasthenia gravis is either an autoimmune or congenital neuromuscular disorder that leads to fluctuating muscle weakness and fatigue. In the most common cases, muscle weakness is caused by circulating antibodies that block acetylcholine (ACh) receptors at the postsynaptic neuromuscular junction, inhibiting the excitatory effects of the neurotransmitter ACh on nicotinic ACh-receptors at neuromuscular junctions (Gilhus, N. E., Owe, J. F., Hoff, J. M., Romi, F., Skeie, G. O., Aarli, J. A. Myasthenia Gravis: A Review of Available Treatment Approaches, Autoimmune Diseases, 2011, Article ID 84739). Lambert-Eaton myasthenic syndrome (also known as LEMS, Lambert-Eaton syndrome, or Eaton-Lambert syndrome) is a rare autoimmune disorder that is characterized by muscle weakness of the limbs. It is the result of an autoimmune reaction in which antibodies are formed against presynaptic voltage-gated calcium channels, and likely other nerve terminal proteins, in the neuromuscular junction. Thirty to fifty percent of patients with acetylcholine receptor (AChR) antibody-negative myasthenia gravis (MG) have antibodies to muscle specific kinase (MuSK) and are referred to as having MuSK-MG (Borges, L. S., Richman, D. P., Muscle-Specific Kinase Myasthenia Gravis, Frontiers in Immunology, 2020, 11, 707). In one embodiment of the present disclosure the neuromuscular disorder is myasthenia gravis. In another embodiment the neuromuscular disorder is autoimmune myasthenia gravis. In another embodiment the neuromuscular disorder is MuSK-MG. In another embodiment the neuromuscular disorder is Lambert-Eaton syndrome. In another embodiment the neuromuscular disorder is seronegative myasthenia gravis.

X-linked myotubular myopathy is a part of a group of centronuclear myopathies where cell nuclei are abnormally located in the centre of muscle cells instead of their normal location at the periphery. It is one of the severest congenital muscle diseases and is characterised by marked muscle weakness, hypotonia and feeding and breathing difficulties (Dowling J J, Lawlor M W, Das S. X-Linked Myotubular Myopathy. 2002 Feb. 25 [Updated 2018 Aug. 23]. In: Adam M P, Ardinger H H, Pagon R A, et al., editors. GeneReviews® [Internet]. Seattle (WA): University of Washington, Seattle; 1993-2022. Available from: https://www.ncbi.nlm.nih.gov/books/NBK1432/). In one embodiment the neuromuscular disorder is myotubular myopathy.

Duchenne muscular dystrophy is a severe type of muscular dystrophy that primarily affects boys resulting initially in fatigue and muscle weakness (Angelini C, Tasca E, Fatigue in muscular dystrophies, Neuromuscular Disorders, 2012, 22 Suppl 3: S214-20). In one embodiment the neuromuscular disorder is Duchenne muscular dystrophy.

Multiple sclerosis (MS) is the most common demyelinating disease, in which the insulating covers of nerve cells in the brain and spinal cord are damaged. This damage disrupts the ability of parts of the nervous system to transmit signals, resulting in a range of signs and symptoms, including physical, mental, and sometimes psychiatric problems. It has been shown that people with multiple sclerosis typically experience greater levels of exercise-induced fatigue compared with healthy individuals (Brotherton et al, J. Neuorophysiol. 2022, 128, 105-117). There are four types of MS in what is known as the Lublin classification: clinically isolated syndrome (CIS), relapsing-remitting MS (RRMS), primary progressive MS (PPMS) and secondary progressive MS (SPMS). In one embodiment, the neuromuscular disorder is selected from the group consisting of multiple sclerosis (MS), clinically isolated syndrome (CIS), relapsing-remitting MS (RRMS), primary progressive MS (PPMS) and secondary progressive MS (SPMS).

Neuromuscular blockade is used in connection with surgery under general anaesthesia. Reversing agents are used for more rapid and safer recovery of muscle function after such blockade. Complications with excessive muscle weakness after blockade during surgery can result in delayed weaning from mechanical ventilation and respiratory complications after the surgery. These complications can have pronounced effects on outcome of the surgery and future quality of life of patients, there is a need for improved reversing agents (Murphy G. S., Brull S. J. Residual neuromuscular block: lessons unlearned. Part I: definitions, incidence, and adverse physiologic effects of residual neuromuscular block. Anaesth Analg. 2010, 111, 120-128). Thus, in one embodiment, the neuromuscular disorder has been induced by a neuromuscular blocking agent. In one particular embodiment the neuromuscular disorder is muscle weakness caused by neuromuscular blockade after surgery. In another embodiment of the present disclosure the compound or the compound for use is used for reversing and/or ameliorating neuromuscular blockade after surgery. In one embodiment, the neuromuscular blockade is drug induced. In one embodiment, the neuromuscular blockade is caused by non-depolarizing neuromuscular blocker or antibiotic agent. In one embodiment the neuromuscular blockade is induced by an antibiotic. In one embodiment the neuromuscular blockade is induced by a non-depolarizing neuromuscular blocker.

In one embodiment the compound or the compound for use of the present disclosure is used to prevent a neuromuscular disorder. The compound or the compound for use may for example be used prophylactically against nerve gas that is known to cause symptoms of muscle weakness and fatigue (Kawamura, Y., Kihara, M., Nishimoto, K., Taki, M. Efficacy of a half dose of oral pyridostigmine in the treatment of chronic fatigue syndrome: three case reports. Pathophysiol., 2003, 9, 189-194). In one embodiment the compound or the compound for use of the present disclosure may be used in the treatment of muscle weakness (such as drooping eyelids, loss of facial expression, constipation, muscle weakness in arms, muscle weakness in legs and dyspnoea) caused by botulism poisoning. In one embodiment the compound or the compound for use of the present disclosure may be used in the treatment of snake bites where the snake toxin, such as α-neurotoxin or myotoxin, is known to cause symptoms of muscle weakness and fatigue.

In one embodiment, the neuromuscular disorder is selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

### Pharmaceutical Formulations

In one embodiment, a composition comprising the compound or the compound for use, according to the present disclosure, is provided. The composition according to the present disclosure may be used for treating, ameliorating and/or preventing a neuromuscular disorder, and/or for use in reversing and/or ameliorating a neuromuscular blockade. Thus, the compositions and compounds described herein can be pharmaceutically acceptable. In one embodiment the composition as described herein is in the form of a pharmaceutical formulation. In one embodiment, the composition as described herein further comprises a pharmaceutically acceptable carrier. In one aspect, the present invention concerns a composition comprising the compound as defined herein and a pharmaceutically acceptable carrier.

### Combination Therapy

The composition of the present disclosure may comprise further active ingredients/agents or other components to increase the efficiency of the composition. Thus, in one embodiment the composition further comprises at least one further active agent. It is appreciated that the active agent can be suitable for treating, preventing or ameliorating said neuromuscular disorder.

The active agent in certain embodiments can be an acetylcholine esterase inhibitor. Said acetylcholine esterase inhibitor may for example be selected from the group consisting of delta-9-tetrahydrocannabinol, carbamates, physostigmine, neostigmine, pyridostigmine, ambenonium, demecarium, rivastigmine, phenanthrene derivatives, galantamine, piperidines, donepezil, tacrine, edrophonium, huperzine, ladostigil, ungeremine and lactucopicrin.

In certain embodiments, the acetylcholine esterase inhibitor is selected from the group consisting of neostigmine, physostigmine and pyridostigmine. In certain embodiments, the acetylcholine esterase inhibitor is neostigmine or pyridostigmine.

The active agent may also be an immunosuppressive drug. Immunosuppressive drugs are drugs that suppress or reduce the strength of the body's immune system. Immunosuppressive drugs include but are not limited to glucocorticoids, corticosteroids, cytostatics, antibodies and drugs acting on immunophilins. In one embodiment the active agent is prednisone.

The active agent may also be an agent that is used in anti-myotonic treatment. Such agents include for example blockers of voltage gated Na⁺ channels, and aminoglycosides.

The active agent may also be an agent for reversing a neuromuscular blockade after surgery. Such agents include for example neostigmine or sugammadex (Org 25969, tradename Bridion).

The active agent may also be an agent for increasing ACh release by blocking voltage-gated K⁺ channels in the pre-synaptic terminal. Such agent includes 3,4-diaminopyridine (Amifampridine; tradename Firdapse).

The active agent may also be an agent for increasing the levels of survival motor neuron (SMN) protein that are produced. For example, by alternating the splicing of the SMN2 gene in order to increase the expression of full-length SMN protein from SMN2 (Zanetta C, Nizzardo M, Simone C, Monguzzi E, Bresolin N, Comi G P, Corti S, “Molecular therapeutic strategies for spinal muscular atrophies: current and future clinical trials”. Clinical Therapeutics, 2014, 36, 128-140). Such agents include antisense oligonucleotides such as Nusinersen (tradename Spinraza) or small molecules such as Risdiplam (tradename Evrysdi).

The active agent may be a gene therapy, for example by using viral vectors to deliver the SMN1 transgene to the affected motor neurons, where it leads to an increase in SMN protein production. Such gene therapies include onasemnogene abeparvovec (tradename Zolgensma). Such gene therapies include nusinersen (tradename Spinraza), risdiplam (tradename Evrysdi) and Branaplam.

The active agent may be a small molecule that increases expression of the SMN2 gene, thus increasing the amount of full-length SMN protein available. Such therapies include salbutamol (also, called albuterol; tradename Ventolin),

The active agent may also be an agent for increasing muscle reactivity. Such agents include skeletal troponin activators such as Tirasemtiv and Reldesemtiv (CK-2127107) (Hwee, D. T., Kennedy, A. R., Hartman, J. J., Ryans, J., Durham, N., Malik, F. I., Jasper, J. R. The small-molecule fast skeletal troponin activator, CK-2127107, improves exercise tolerance in a rat model of heart failure. J. Pharmacol. Exp. Ther., 2015, 353, 159-168). Such agents may also be antibodies that block the activation of the skeletal muscle protein myostatin, such as Apitegromab (SRK-015) or GYM329 (RO7204239),

The active agent may also be an agent that disrupts or blocks the IgG-FcRn interaction thereby reducing the overall IgG recycling. Such agents may be antibodies, such as the aglycosylated immunoglobulin (Ig)G1 monoclonal antibody Nipocalimab, or IgG1 Fc fragment such as Efgartigimod alfa (tradename Vyvgart).

The active agent may also be an agent that is an inhibitor of the complement component C5a. The active agent may also be an agent that downregulates the overexpression of PMP22 protein, leading to improvement of neuronal signalling in dysfunctional peripheral nerves. Such agents may be combination drugs such as PXT3003. Alternatively, the active agent may also be an agent that binds to the protein complement component 5 (C5) and inhibits its cleavage into C5a and C5b. Such agents may be Zilucoplan (RA101495).

### Methods

In one aspect, the present invention relates to a compound as defined herein for use as a medicament.

In one aspect, the present disclosure relates to a compound of Formula (I):

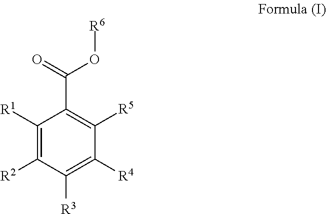

wherein:
   R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.

In one embodiment, R¹ is F. In one embodiment, R¹ is Cl. In one embodiment, R¹ is Br. In one embodiment, R¹ is I. In one embodiment, R¹ is C₁₋₃ alkyl optionally substituted with one or more, identical or different, substituents R⁸. In one embodiment, R¹ is —OC₁₋₃ alkyl optionally substituted with one or more, identical or different, substituents R⁷. In one embodiment, R¹ is —OC₃₋₅ cycloalkyl optionally substituted with one or more, identical or different, substituents R⁷. In one embodiment, R¹ is —SC₁₋₃ alkyl optionally substituted with one or more, identical or different, substituents R⁷. In one embodiment, R¹ is —SC₃₋₅ cycloalkyl optionally substituted with one or more, identical or different, substituents R⁷.

In one embodiment, R¹ is selected from the group consisting of F; Cl; Br; C₁₋₃ alkyl optionally substituted with one or more, identical or different, substituents R⁸ and —OC₁₋₃ alkyl optionally substituted with one or more, identical or different, substituents R⁷. In one embodiment, R¹ is selected from the group consisting of F, Cl, Br, C₁₋₃ alkyl optionally substituted with one or more, identical or different, substituents R⁸. In one embodiment, R¹ is selected from the group consisting of MeO, Et, Me or Cl. In one embodiment, R¹ is selected from the group consisting of Et, Me or Cl. In one embodiment, R¹ is Me.

In one embodiment, if R¹ is F; Cl; Br or I then R² is not F; Cl; Br or I. In one embodiment, R² is C₁₋₃ alkyl optionally substituted with one or more, identical or different, substituents R⁸. In one embodiment, R² is Cl or Me. In one embodiment, R² is Me. In one embodiment, R² is C₂₋₃ alkenyl optionally substituted with one or more, identical or different, substituents R⁸. In one embodiment, R² is F. In one embodiment, R² is Cl. In one embodiment, R² is Br. In one embodiment, R² is I. In one embodiment, R³ is H. In one embodiment, R³ is F.

In one embodiment, R⁴ is selected from the group consisting of F, Cl and Br. In one embodiment, R⁴ is selected from the group consisting of F and Cl. In one embodiment, R⁴ is Cl. In one embodiment, R⁴ is F. In one embodiment, R⁴ is Br.

In one embodiment, R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷ and —SC₁₋₃ alkyl optionally substituted with one or more, identical or different, substituents R⁷. In one embodiment, R⁵ is selected from the group consisting of —OC₁₋₃ alkyl optionally substituted with one or more, identical or different, substituents R⁷ and —OC₃₋₅ cycloalkyl optionally substituted with one or more, identical or different, substituents R⁷.

In one embodiment, R⁵ is selected from the group consisting of —OC₁₋₃ alkyl optionally substituted with one or more, identical or different, substituents R⁷ and —SC₁₋₃ alkyl optionally substituted with one or more, identical or different, substituents R⁷. In one embodiment, R⁵ is selected from the group consisting of —OC₁₋₃ alkyl optionally substituted with one or more, identical or different, substituents R⁷. In one embodiment, R⁵ is selected from the group consisting of —SC₁₋₃ alkyl optionally substituted with one or more, identical or different, substituents R⁷. In one embodiment, R⁵ is selected from the group consisting of —OC₃₋₅ cycloalkyl optionally substituted with one or more, identical or different, substituents R⁷. In one embodiment, R⁵ is selected from the group consisting of —SC₃₋₅ cycloalkyl optionally substituted with one or more, identical or different, substituents R⁷. In one embodiment, R⁵ is selected from the group consisting of cyclopropoxy, —OEt, —OMe, —OCH₂F, —OCHF₂, —OCF₃, —SMe, —SCH₂F, —SCHF₂ and —SCF₃. In one embodiment, R⁵ is selected from the group consisting of —OMe, —OCH₂F, —OCHF₂, —OCF₃ and —SMe.

In one embodiment, R⁶ is H.

In one embodiment, R⁷ is F. In one embodiment, R⁷ is D. In one embodiment, R⁷ is Cl.

In one embodiment, R⁸ is D. In one embodiment, R⁸ is F. In one embodiment, R⁸ is Cl.

In one embodiment, R⁸ is SMe. In one embodiment, R⁸ is OMe.

In one embodiment, the EC₅₀ of the compound is <10 μM, such as <5 μM, such as <2 μM, and such as <1 μM.

In one aspect, the disclosure concerns a compound of Formula (I):

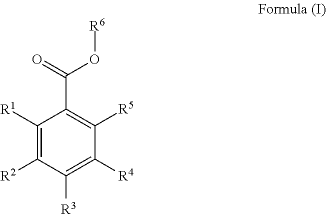

wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.

In one embodiment, when R¹ is F or Me, R² is Me, R³ is H, R⁴ is Cl or Br and R⁵ is OMe, then R⁶ is not H, Me or Et. In one embodiment, when R¹ is Cl or Br, R² is Me, R³ is H, R⁴ is F and R⁵ is OMe, then R⁶ is not H, Me or Et. In one embodiment, when R¹ is F, R² is Me, R³ is F, R⁴ is F and R⁵ is OMe, then R⁶ is not H.

In one aspect, the disclosure concerns a compound of Formula (I):

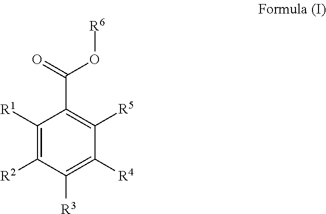

wherein:
   R¹ is selected from the group consisting of F; Cl; Br; C₁₋₃ alkyl optionally substituted with one or more, identical or different, substituents R⁸;
   R² is selected from the group consisting of C₁₋₃ alkyl optionally substituted with one or more, identical or different, substituents R⁸;
   R³ is H;
   R⁴ is selected from the group consisting of F, Cl and Br;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷;
   R⁶ is H;
   R⁷ is F; and
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.
In one aspect, when R¹ is F or Me, R² is Me, R³ is H, R⁴ is Cl or Br and R⁵ is OMe, then R⁶ is not H. In one aspect, when R¹ is Cl or Br, R² is Me, R³ is H, R⁴ is F and R⁵ is OMe, then R⁶ is not H.

In one aspect, the compound for use in treating, ameliorating and/or preventing a neuromuscular disorder, and/or for use in reversing and/or ameliorating a neuromuscular blockade is selected from the group consisting of:
3-bromo-5-fluoro-2,6-dimethoxybenzoic acid;
3-fluoro-2,6-dimethoxy-5-methylbenzoic acid;
5-chloro-2-methyl-3-[(methylsulphanyl)methyl]benzoic acid;
3,5-dichloro-2,6-dimethoxybenzoic acid;
3-chloro-2-cyclopropoxy-5,6-dimethylbenzoic acid;
2,5-dichloro-6-methoxy-3-methylbenzoic acid;
3-chloro-2,6-dimethoxy-5-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid;
3-chloro-2-ethoxy-5,6-dimethylbenzoic acid;
3-bromo-2-methoxy-5,6-dimethylbenzoic acid; and
3-chloro-2-methoxy-5,6-dimethylbenzoic acid.

In one aspect, the present disclosure relates to a method of treating, preventing and/or ameliorating a neuromuscular disorder, said method comprising administering a therapeutically effective amount of the compound or the compound for use as defined herein to a person in need thereof.

In one aspect, the present disclosure relates to a method of reversing and/or ameliorating a neuromuscular blockade, said method comprising administering a therapeutically effective amount of the compound or the compound for use as defined herein to a person in need thereof.

In one aspect, the present disclosure relates to a method for treating and/or ameliorating a neuromuscular disorder, or for reversing and/or ameliorating a neuromuscular blockade comprising administering to a patient in need thereof a therapeutically effective amount of a compound of Formula (I):

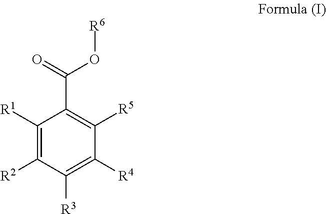

wherein:
   R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;

or a pharmaceutically acceptable salt, hydrate, polymorph, tautomer, or solvate thereof.

In one aspect, the present invention related to a compound as defined herein for use in the treatment of an indication selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.

In one aspect, the present disclosure relates to a method for recovery of neuromuscular transmission, said method comprising administering a therapeutically effective amount of the compound or the compound for use as defined herein to a person in need thereof.

The person in need thereof may be a person having a neuromuscular disorder or a person at risk of developing a neuromuscular disorder or a person having symptoms of muscle weakness and/or fatigue. In another embodiment the person in need thereof is a person with reduced neuromuscular transmission safety with prolonged recovery after neuromuscular blockade. Types of neuromuscular disorders are defined herein above. In an embodiment the person has amyotrophic lateral sclerosis, spinal muscular atrophy, myasthenia gravis or Lambert-Eaton syndrome.

A therapeutically effective amount is an amount that produces a therapeutic response or desired effect in the person taking it. Administration routes, formulations and dosages can be optimized by persons of skill in the art.

The method of treatment may be combined with other methods that are known to treat, prevent and/or ameliorate neuromuscular disorders. The treatment method may for example be combined with administration of any of the agents mentioned herein above. In one embodiment the treatment is combined with administration of acetylcholine esterase inhibitor such as for example neostigmine or pyridostigmine.

In one aspect, the invention relates to a method for recovery of force in muscles with neuromuscular dysfunction, said method comprising administering a compound or a composition as defined herein to a subject in need thereof. The term “recovery of force in muscles with neuromuscular dysfunction” as used herein refers to the ability of a compound to recover contractile force in nerve-stimulated healthy rat muscle after exposure to submaximal concentration (115 nM) of tubocurarine for 90 mins. Recovery of force is quantified as the percentage of the force prior to tubocurarine that is recovered after addition of the compound. In one embodiment, said recovery of force is >5%, such as >10%, such as >15%, such as >20%, such as >25%, such as >30%, such as >35%.

In one aspect, the present disclosure relates use of a compound of Formula (I):

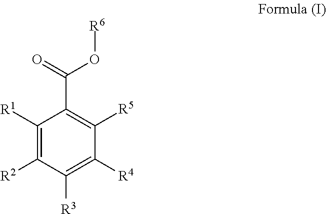

wherein:
   R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;

or a pharmaceutically acceptable salt, hydrate, polymorph, tautomer, or solvate thereof for the manufacture of a medicament for the treatment, amelioration and/or prevention of a neuromuscular disorder, and/or for reversing and/or ameliorating a neuromuscular blockade.

One aspect of the disclosure relates to use of a compound as defined herein, for the manufacture of a medicament for the treatment, prevention and/or amelioration of a neuromuscular disorder.

Another aspect relates to use of a compound as defined herein, for the manufacture of a medicament or a reversal agent for reversing and/or ameliorating a neuromuscular blockade after surgery.

### Method of Manufacturing

In one aspect, the present disclosure relates to methods of manufacturing compounds or compounds for use according to formula (I).

Compounds according to the present invention may be prepared according to any conventional methods of chemical synthesis known by the skilled person, e.g. those described in the working examples. The starting materials for the processes described in the present application are known or may readily be prepared by conventional methods known by the skilled artisan from commercially available chemicals.

The end products of the reactions described herein may be isolated by conventional technique such as extraction, crystallisation, distillation, chromatography etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

### Items

1. A compound of Formula (I):

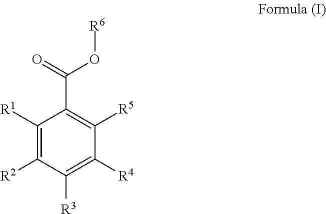

wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H, Me or Et; and with the proviso that when R¹ is Cl or Br, R² is Me, R³ is H, R⁴ is F and R⁵ is OMe, then R⁶ is not H, Me or Et; and with the proviso that when R¹ is F, R² is Me, R³ is F, R⁴ is F and R⁵ is OMe, then R⁶ is not H.
2. The compound according to any one of the preceding items, wherein R¹ is selected from the group consisting of F, Cl, Br, and C₁₋₃ alkyl optionally substituted with one or more, identical or different, substituents R⁸.
3. The compound according to any one of the preceding items, wherein R¹ is selected from the group consisting of Et, Me or Cl.
4. The compound according to any one of the preceding items, wherein R¹ is Me.
5. The compound according to any one of the preceding items, wherein R² is C₁₋₃ alkyl optionally substituted with one or more, identical or different, substituents R⁸.
6. The compound according to any one of the preceding items, wherein R² is Me.
7. The compound according to any one of the preceding items, wherein R³ is H.
8. The compound according to any one of the preceding items, wherein R⁴ is selected from the group consisting of F, Cl and Br.
9. The compound according to any one of the preceding items, wherein R⁴ is selected from the group consisting of F and Cl.
10. The compound according to any one of the preceding items, wherein R⁴ is Cl.
11. The compound according to any one of the preceding items, wherein R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷ and —SC₁₋₃ alkyl optionally substituted with one or more, identical or different, substituents R⁷.
12. The compound according to any one of the preceding items, wherein R⁵ is selected from the group consisting of —OC₁₋₃ alkyl optionally substituted with one or more, identical or different, substituents R⁷ and —OC₃₋₅ cycloalkyl optionally substituted with one or more, identical or different, substituents R⁷.
13. The compound according to any one of the preceding items, wherein R⁵ is selected from the group consisting of —OC₁₋₃ alkyl optionally substituted with one or more, identical or different, substituents R⁷ and —SC₁₋₃ alkyl optionally substituted with one or more, identical or different, substituents R⁷.
14. The compound according to any one of the preceding items, wherein R⁵ is selected from the group consisting of —OC₁₋₃ alkyl optionally substituted with one or more, identical or different, substituents R⁷.
15. The compound according to any one of the preceding items, wherein R⁵ is selected from the group consisting of cyclopropoxy, —OEt, —OMe, —OCH₂F, —OCHF₂, —OCF₃, —SMe, —SCH₂F, —SCHF₂ and —SCF₃.
16. The compound according to any one of the preceding items, wherein R⁵ is selected from the group consisting of —OMe, —OCH₂F, —OCHF₂, —OCF₃ and —SMe.
17. The compound according to any one of the preceding items, wherein R⁶ is H.
18. The compound according to any one of the preceding items, wherein R⁷ is F.
19. The compound according to any one of the preceding items, wherein R⁸ is SMe.
20. The compound according to any one of the preceding items, wherein the EC₅₀ of the compound is <10 μM, such as <5 μM, such as <2 μM, and such as <1 μM.
21. The compound according to any one of the preceding items, wherein
   R¹ is selected from the group consisting of F; Cl; Br and C₁₋₃ alkyl optionally substituted with one or more, identical or different, substituents R⁸;
   R² is selected from the group consisting of C₁₋₃ alkyl optionally substituted with one or more, identical or different, substituents R⁸;
   R³ is H;
   R⁴ is selected from the group consisting of F, Cl and Br;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; and —SC₁₋₃ alkyl optionally substituted with one or more, identical or different, substituents R⁷;
   R⁶ is H;
   R⁷ is F; and
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl;
   or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H; and with the proviso that when R¹ is Cl or Br, R² is Me, R³ is H, R⁴ is F and R⁵ is OMe, then R⁶ is not H.
22. The compound according to any one of the preceding items, wherein the compound is selected from the group consisting of:
3-chloro-2-cyclopropoxy-5,6-dimethylbenzoic acid;
2,5-dichloro-6-methoxy-3-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid; and
3-chloro-2-ethoxy-5,6-dimethylbenzoic acid.
23. The compound according to any one of the preceding items, wherein the compound has activity on the CIC-1 receptor.
24. The compound according to any one of the preceding items, wherein the compound is an inhibitor of the CIC-1 ion channel.
25. The compound according to any one of the preceding items, wherein the recovery of force in muscles with neuromuscular dysfunction is >5%, for example >10%, for example >15%, for example >20%, for example >25%, for example >30% and for example >35%.
26. The compound according to any one of the preceding items, wherein the compound improves the recovered force in isolated rat soleus muscles after exposure to tubocurarine.
27. The compound according to any one of the preceding items for use as a medicament.
28. A composition comprising the compound according to any one of the preceding items.
29. The composition according to any one of the preceding items, wherein the composition further comprises a pharmaceutically acceptable carrier.
30. The composition according to any one of items 28 to 29, wherein the composition further comprises at least one further active agent.
31. The composition according to item 30, wherein said further active agent is suitable for treating, preventing or ameliorating said neuromuscular disorder.
32. The compound according to any one of items 1 to 27 or composition according to any one of items 28 to 31 for use in the treatment of an indication selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder and multifocal motor neuropathy.
33. The compound according to any one of items 1 to 27 or composition according to any one of items 28 to 31 for use in the treatment of sarcopenia.
34. The compound according to any one of items 1 to 27 or composition according to any one of items 28 to 31 for use in reversing and/or ameliorating a neuromuscular blockade.
35. The compound according to any one of items 1 to 27 or composition according to any one of items 28 to 31 for use in the treatment of botulism poisoning, in the treatment of snake bites, in the treatment of nerve gas poisoning or prophylactically against nerve gas poisoning.
36. A compound of Formula (I):

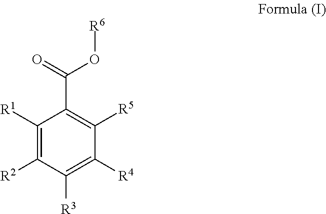

wherein:
   R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.
37. The compound for use according to item 36, wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
      R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
      R³ is selected from the group consisting of H and F;
      R⁴ is selected from the group consisting of F, Cl, Br and I;
      R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
      R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
      R⁷ is independently selected from the group consisting of deuterium, F and Cl;
      R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
      R⁹ is independently selected from the group consisting of deuterium, methoxy, nitro, cyano, Cl, Br, I, and F;
   or a pharmaceutically acceptable salt, hydrate, polymorph, tautomer, or solvate thereof.
38. The compound for use according to item 37, with the proviso that when R¹ is F or Me, R² is Me, R³ is H, R⁴ is Cl or Br and R⁵ is OMe, then R⁶ is not H, Me or Et and with the proviso that when R¹ is Cl or Br, R² is Me, R³ is H, R⁴ is F and R⁵ is OMe, then R⁶ is not H, Me or Et and with the proviso that when R¹ is F, R² is Me, R³ is F, R⁴ is F and R⁵ is OMe, then R⁶ is not H.
39. The compound for use according to item 36, wherein:
   R¹ is selected from the group consisting of F; Cl; Br; C₁₋₃ alkyl optionally substituted with one or more, identical or different, substituents R⁸;
   R² is selected from the group consisting of C₁₋₃ alkyl optionally substituted with one or more, identical or different, substituents R⁸;
   R³ is H;
   R⁴ is selected from the group consisting of F, Cl and Br;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷;
   R⁶ is H;
   R⁷ is F; and
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl;
   or a pharmaceutically acceptable salt, hydrate, polymorph, tautomer, or solvate thereof.
40. The compound for use according to item 39, with the proviso that when R¹ is F or Me, R² is Me, R³ is H, R⁴ is Cl or Br and R⁵ is OMe, then R⁶ is not H and with the proviso that when R¹ is Cl or Br, R² is Me, R³ is H, R⁴ is F and R⁵ is OMe, then R⁶ is not H.
41. The compound for use according to any one of the preceding items, wherein R¹ is selected from the group consisting of F; Cl; Br; C₁₋₃ alkyl optionally substituted with one or more, identical or different, substituents R⁸ and —OC₁₋₃ alkyl optionally substituted with one or more, identical or different, substituents R⁷
42. The compound for use according to any one of the preceding items, wherein R¹ is selected from the group consisting of F, Cl, Br, C₁₋₃ alkyl optionally substituted with one or more, identical or different, substituents R⁸
43. The compound for use according to any one of the preceding items, wherein R¹ is selected from the group consisting of MeO, Et, Me or Cl
44. The compound for use according to any one of the preceding items, wherein R¹ is selected from the group consisting of Et, Me or Cl
45. The compound for use according to any one of the preceding items, wherein R¹ is Me.
46. The compound for use according to any one of the preceding items, wherein if R¹ is F; Cl; Br or I then R² is not F; Cl; Br or I.
47. The compound for use according to any one of the preceding items, wherein R² is C₁₋₃ alkyl optionally substituted with one or more, identical or different, substituents R⁸
48. The compound for use according to any one of the preceding items, wherein R² is Cl or Me
49. The compound for use according to any one of the preceding items, wherein R² is Me.
50. The compound for use according to any one of the preceding items, wherein R³ is H.
51. The compound for use according to any one of the preceding items, wherein R⁴ is selected from the group consisting of F, Cl and Br
52. The compound for use according to any one of the preceding items, wherein R⁴ is selected from the group consisting of F and Cl
53. The compound for use according to any one of the preceding items, wherein R⁴ is Cl.
54. The compound for use according to any one of the preceding items, wherein R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷ and —SC₁₋₃ alkyl optionally substituted with one or more, identical or different, substituents R⁷
55. The compound for use according to any one of the preceding items, wherein R⁵ is selected from the group consisting of —OC₁₋₃ alkyl optionally substituted with one or more, identical or different, substituents R⁷ and —OC₃₋₅ cycloalkyl optionally substituted with one or more, identical or different, substituents R⁷.
56. The compound for use according to any one of the preceding items, wherein R⁵ is selected from the group consisting of —OC₁₋₃ alkyl optionally substituted with one or more, identical or different, substituents R⁷ and —SC₁₋₃ alkyl optionally substituted with one or more, identical or different, substituents R⁷
57. The compound for use according to any one of the preceding items, wherein R⁵ is selected from the group consisting of —OC₁₋₃ alkyl optionally substituted with one or more, identical or different, substituents R⁷
58. The compound for use according to any one of the preceding items, wherein R⁵ is selected from the group consisting of cyclopropoxy, —OEt, —OMe, —OCH₂F, —OCHF₂, —OCF₃, —SMe, —SCH₂F, —SCHF₂ and —SCF₃
59. The compound for use according to any one of the preceding items, wherein R⁵ is selected from the group consisting of —OMe, —OCH₂F, —OCHF₂, —OCF₃ and —SMe.
60. The compound for use according to any one of the preceding items, wherein R⁶ is H.
61. The compound for use according to any one of the preceding items, wherein R⁷ is F.
62. The compound for use according to any one of the preceding items, wherein R⁸ is SMe.
63. The compound for use according to any one of the preceding items, wherein the compound is selected from the group consisting of:
3-bromo-5-fluoro-2,6-dimethoxybenzoic acid;
3-fluoro-2,6-dimethoxy-5-methylbenzoic acid;
5-chloro-2-methyl-3-[(methylsulphanyl)methyl]benzoic acid;
3,5-dichloro-2,6-dimethoxybenzoic acid;
3-chloro-2-cyclopropoxy-5,6-dimethylbenzoic acid;
2,5-dichloro-6-methoxy-3-methylbenzoic acid;
3-chloro-2,6-dimethoxy-5-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid;
3-chloro-2-ethoxy-5,6-dimethylbenzoic acid;
3-bromo-2-methoxy-5,6-dimethylbenzoic acid; and
3-chloro-2-methoxy-5,6-dimethylbenzoic acid.
64. The compound for use according to any one of the preceding items in the treatment of an indication selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.
65. The compound for use according to any one of the preceding items in the treatment of sarcopenia.
66. The compound for use according to any one of the preceding items in reversing and/or ameliorating a neuromuscular blockade wherein the neuromuscular blockade has been induced by a neuromuscular blocking agent.
67. The compound for use according to any one of the preceding items in the treatment of botulism poisoning, in the treatment of snake bites, in the treatment of nerve gas poisoning or prophylactically against nerve gas poisoning.
68. A method for treating and/or ameliorating a neuromuscular disorder, or for reversing and/or ameliorating a neuromuscular blockade comprising administering to a patient a therapeutically effective amount of a compound of Formula (I):

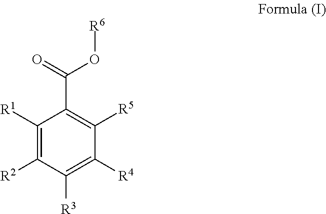

wherein:
   R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
   R⁷ is independently selected from the group consisting of deuterium, F and Cl;
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
   R⁹ is independently selected from the group consisting of deuterium, methoxy, nitro, cyano, Cl, Br, I, and F;
or a pharmaceutically acceptable salt, hydrate, polymorph, tautomer, or solvate thereof.
69. The method according to item 68, wherein:
   R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
   R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
   R³ is selected from the group consisting of H and F;
   R⁴ is selected from the group consisting of F, Cl, Br and I;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
      R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
      R⁷ is independently selected from the group consisting of deuterium, F and Cl;
      R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
      R⁹ is independently selected from the group consisting of deuterium, methoxy, nitro, cyano, Cl, Br, I, and F;
   or a pharmaceutically acceptable salt, hydrate, polymorph, tautomer, or solvate thereof.
70. The method according to any one of items 68 or 69, wherein:
   R¹ is selected from the group consisting of F; Cl; Br; C₁₋₃ alkyl optionally substituted with one or more, identical or different, substituents R⁸;
   R² is selected from the group consisting of C₁₋₃ alkyl optionally substituted with one or more, identical or different, substituents R⁸;
   R³ is H;
   R⁴ is selected from the group consisting of F, Cl and Br;
   R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷;
   R⁶ is H;
   R⁷ is F; and
   R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl;
   or a pharmaceutically acceptable salt, hydrate, polymorph, tautomer, or solvate thereof.
71. The method according to any one of items 68, 69 or 70, wherein R¹ is selected from the group consisting of F; Cl; Br; C₁₋₃ alkyl optionally substituted with one or more, identical or different, substituents R⁸ and —OC₁₋₃ alkyl optionally substituted with one or more, identical or different, substituents R⁷
72. The method according to any one of items 68, 69 or 70, wherein R¹ is selected from the group consisting of F, Cl, Br, C₁₋₃ alkyl optionally substituted with one or more, identical or different, substituents R⁸
73. The method according to any one of items 68, 69 or 70, wherein R¹ is selected from the group consisting of MeO, Et, Me or Cl
74. The method according to any one of items 68, 69 or 70, wherein R¹ is selected from the group consisting of Et, Me or Cl
75. The method according to any one of items 68, 69 or 70, wherein R¹ is Me.
76. The method according to any one of items 68, 69, 70, 71, 72, 73, 74 or 75, wherein if R¹ is F; Cl; Br or I then R² is not F; Cl; Br or I.
77. The method according to any one of items 68, 69, 70, 71, 72, 73, 74, 75 or 76, wherein R² is C₁₋₃ alkyl optionally substituted with one or more, identical or different, substituents R⁸
78. The method according to any one of items 68, 69, 70, 71, 72, 73, 74, 75 or 76, wherein R² is Cl or Me
79. The method according to any one of items 68, 69, 70, 71, 72, 73, 74, 75 or 76, wherein R² is Me.
80. The method according to any one of items 68, 69, 70, 71, 72, 73, 74, 75, 76, 77, 78 or 79, wherein R³ is H.
81. The method according to any one of items 68, 69, 70, 71, 72, 73, 74, 75, 76, 77, 78, 79 or 80, wherein R⁴ is selected from the group consisting of F, Cl and Br
82. The method according to any one of items 68, 69, 70, 71, 72, 73, 74, 75, 76, 77, 78, 79 or 80, wherein R⁴ is selected from the group consisting of F and Cl
83. The method according to any one of items 68, 69, 70, 71, 72, 73, 74, 75, 76, 77, 78, 79 or 80, wherein R⁴ is Cl.
84. The method according to any one of items 68, 69, 70, 71, 72, 73, 74, 75, 76, 77, 78, 79, 80, 81, 82 or 83, wherein R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷ and —SC₁₋₃ alkyl optionally substituted with one or more, identical or different, substituents R⁷
85. The method according to any one of items 68, 69, 70, 71, 72, 73, 74, 75, 76, 77, 78, 79, 80, 81, 82 or 83, wherein R⁵ is selected from the group consisting of —OC₁₋₃ alkyl optionally substituted with one or more, identical or different, substituents R⁷ and —OC₃₋₅ cycloalkyl optionally substituted with one or more, identical or different, substituents R⁷.
86. The method according to any one of items 68, 69, 70, 71, 72, 73, 74, 75, 76, 77, 78, 79, 80, 81, 82 or 83, wherein R⁵ is selected from the group consisting of —OC₁₋₃ alkyl optionally substituted with one or more, identical or different, substituents R⁷ and —SC₁₋₃ alkyl optionally substituted with one or more, identical or different, substituents R⁷
87. The method according to any one of items 68, 69, 70, 71, 72, 73, 74, 75, 76, 77, 78, 79, 80, 81, 82 or 83, wherein R⁵ is selected from the group consisting of —OC₁₋₃ alkyl optionally substituted with one or more, identical or different, substituents R⁷
88. The method according to any one of items 68, 69, 70, 71, 72, 73, 74, 75, 76, 77, 78, 79, 80, 81, 82 or 83, wherein R⁵ is selected from the group consisting of cyclopropoxy,-OEt, —OMe, —OCH₂F, —OCHF₂, —OCF₃, —SMe, —SCH₂F, —SCHF₂ and —SCF₃
89. The method according to any one of items 68, 69, 70, 71, 72, 73, 74, 75, 76, 77, 78, 79, 80, 81, 82 or 83, wherein R⁵ is selected from the group consisting of —OMe, —OCH₂F, —OCHF₂, —OCF₃ and —SMe.
90. The method according to any one of items 68, 69, 70, 71, 72, 73, 74, 75, 76, 77, 78, 79, 80, 81, 82, 83, 84, 85, 86, 87, 88 or 89, wherein R⁶ is H.
91. The method according to any one of items 68, 69, 70, 71, 72, 73, 74, 75, 76, 77, 78, 79, 80, 81, 82, 83, 84, 85, 86, 87, 88, 89 or 90, wherein R⁷ is F.
92. The method according to any one of items 68, 69, 70, 71, 72, 73, 74, 75, 76, 77, 78, 79, 80, 81, 82, 83, 84, 85, 86, 87, 88, 89, 90 or 91, wherein R⁸ is SMe.
93. The method according to any one of items 68, wherein the compound is selected from the group consisting of:
3-bromo-5-fluoro-2,6-dimethoxybenzoic acid;
3-fluoro-2,6-dimethoxy-5-methylbenzoic acid;
5-chloro-2-methyl-3-[(methylsulphanyl)methyl]benzoic acid;
3,5-dichloro-2,6-dimethoxybenzoic acid;
3-chloro-2-cyclopropoxy-5,6-dimethylbenzoic acid;
2,5-dichloro-6-methoxy-3-methylbenzoic acid;
3-chloro-2,6-dimethoxy-5-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid;
3-chloro-2-ethoxy-5,6-dimethylbenzoic acid;
3-bromo-2-methoxy-5,6-dimethylbenzoic acid; and
3-chloro-2-methoxy-5,6-dimethylbenzoic acid.
94. The method according to any one of items 68, 69, 70, 71, 72, 73, 74, 75, 76, 77, 78, 79, 80, 81, 82, 83, 84, 85, 86, 87, 88, 89, 90, 91, 92 or 93, wherein the compound is an inhibitor of the CIC-1 ion channel.
95. The method of item 68, wherein said neuromuscular disorder is one selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis and multifocal motor neuropathy.
96. The method of item 68, wherein said neuromuscular disorder is sarcopenia.
97. The method of item 68, wherein said method is for reversing and/or ameliorating a neuromuscular blockade induced by a neuromuscular blocking agent.
98. The method of item 68, wherein said neuromuscular disorder is due to botulism poisoning, snake bites, nerve gas poisoning or prophylactically against nerve gas poisoning.
99. The compound for use according to any one of the preceding items, wherein said compound is comprised in a composition.
100. The compound for use according to any one of the preceding items, wherein the composition is a pharmaceutical composition.
101. The compound for use according to any one of the preceding items, wherein the composition further comprises a pharmaceutically acceptable carrier.
102. The compound for use according to any one of items, wherein the composition further comprises at least one further active agent.
103. The compound for use according to any one of the preceding items, wherein said further active agent is suitable for treating, preventing or ameliorating said neuromuscular disorder.
104. The compound for use according to any one of the preceding items, wherein said further active agent is an acetylcholine esterase inhibitor.
105. The compound for use according to any one of the preceding items, wherein said acetylcholine esterase inhibitor is selected from the group consisting of delta-9-tetrahydrocannabinol, carbamates, physostigmine, neostigmine, pyridostigmine, ambenonium, demecarium, rivastigmine, phenanthrene derivatives, galantamine, piperidines, donepezil, tacrine, edrophonium, huperzine, ladostigil, ungeremine and lactucopicrin.
106. The compound for use according to any one of the preceding items, wherein said acetylcholine esterase inhibitor is neostigmine or pyridostigmine.
107. The compound for use according to any one of the preceding items, wherein said further active agent is sugammadex.
108. The compound for use according to any one of the preceding items, wherein said further active agent is 3,4-diaminopyridine.
109. A method of treating, preventing and/or ameliorating a neuromuscular disorder, said method comprising administering a therapeutically effective amount of the compound as defined in any one of the preceding items to a person in need thereof.
110. Use of a compound as defined in any one of the preceding items, for the manufacture of a medicament for the treatment, prevention and/or amelioration of a neuromuscular disorder, and/or for reversing and/or ameliorating of a neuromuscular blockade.
111. A method of reversing and/or ameliorating a neuromuscular blockade, said method comprising administering a therapeutically effective amount of the compound as defined in any one of the preceding items to a person in need thereof.
112. A method for recovery of neuromuscular transmission, said method comprising administering a therapeutically effective amount of the compound as defined in any one of the preceding items to a person in need thereof.
113. A method for recovering neuromuscular transmission, the method comprising administering a compound as defined in any one of the preceding items to an individual in need thereof.

### EXAMPLES

### General Synthetic Strategies

General methods for the synthesis of carboxylic acids and substitution on aromatic rings are featured in literature sources such as: March's Advanced Organic Chemistry: Reactions, Mechanisms, and Structure, 8th Edition, Michael B. Smith, Ed.; ISBN: 978-1-119-37179-3; John Wiley, 2020.

### Materials and Methods

### NMR Spectra

¹H-NMR and ¹⁹F-NMR spectra were recorded on a Bruker AM-300 spectrometer and were calibrated using residual nondeuterated solvent as internal reference. Spectra were processed using Spinworks version 4.0 (developed by Dr. Kirk Marat, Department of Chemistry, University of Manitoba).

### LC/MS System

Samples were analysed by direct inject on a Waters Micromass Quattro Ultima with a Waters 2695 HPLC. Mass spectra were recorded in ESI scan mode (negative/positive). Mass spectra were processed using Water MassLynx Mass Spectrometry Software.

### HPLC Method

The product was analysed by a Waters 2695 HPLC consisting of a Waters 996 photodiode array detector (Waters, Milford, MA, USA) and a Kromasil Eternity C18, 5 μm, 4.6×150 mm column. Flow rate: 1 mL/minute, run time 20 minutes. Spectra were processed using Waters Empower Software. The chromatographic system was operated using gradient elution with HPLC grade solvents methanol (solvent A), 0.1% formic acid in water (solvent B), and acetonitrile (solvent C) at ambient temperature and UV detection at either 280 nm or 254 nm. HPLC gradients were either 0% of A to 100% of A over 15 minutes or 10% of C to 100% of C over 15 minutes or gradient 10% of C to 100% of C over 7 minutes.

### Example 1: Synthesis of 3-fluoro-2,6-dimethoxy-5-methylbenzoic acid (A-1)

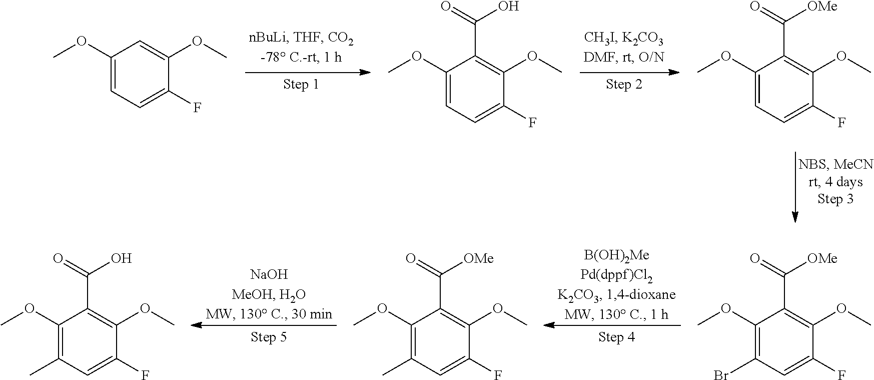

### Step 1: 3-Fluoro-2,6-dianisic acid

To a solution of 1-fluoro-2,4-dimethoxybenzene (1.0 g, 6.40 mmol) in THF (10 mL) at −78° C. was added n-BuLi (2.5 M in hexane) (2.82 mL, 7.04 mmol). The reaction mixture was stirred at that temperature for 1 h, and CO₂ (g) was bubbled through it for 10 min. and stirred for 1 h at −78° C. The temperature of the mixture allowed to reach 0° C. and quenched 1M NaOH until the pH reached ˜11. The reaction mixture was washed with CH₂Cl₂ (2×10 mL) to remove impurities, and the aqueous layer was acidified with 1N HCl to a pH value of ˜1. Extraction with EtOAc (50 mL), drying (Na₂SO₄) filtration and evaporation in vacuo provided 3-fluoro-2,6-dianisic acid (˜1.0 g) which was utilised directly in the next step without further purification.

### Step 2: Methyl 3-fluoro-2,6-dianisate

Potassium carbonate (2.07 g, 14.99 mmol) was introduced to a solution of 3-fluoro-2,6-dianisic acid (1.0 g, 4.99 mmol) in dimethylformamide (15 mL) and the mixture was stirred at ambient temperature for 20 min. Iodomethane (0.467 mL, 7.49 mmol) was added and the reaction mixture stirred for 18 h at ambient temperature. The suspension was quenched with water, extracted with EtOAc (50 mL) and the extract treated with brine (2×20 mL). The organic layer was separated, dried over Na₂SO₄, and concentrated. The crude product was purified by column chromatography on silica gel eluting with (0-30%) EtOAc/hexane to afford methyl 3-fluoro-2,6-dianisate (0.354 g: 33% yield).

¹H NMR (300 MHZ, CDCl₃) δ 7.25 (dd, 1H), 6.74 (dd, 1H), 4.14 (d, 3H), 4.11 (s, 3H), 3.97 (s, 3H).

¹⁹F NMR (300 MHZ, CDCl3) δ −139.78 ppm.

### Step 3: Methyl 3-bromo-5-fluoro-2,6-dianisate

To a solution of methyl 3-fluoro-2,6-dianisate (0.35 g, 1.64 mmol) in CH₃CN (5 mL) was added N-bromosuccinimide (0.32 g, 1.79 mmol) and the reaction mixture was stirred at ambient temperature for 4 days. The reaction mixture was concentrated, extracted with EtOAc (30 mL), washed with water (2×10 mL), and brine (15 mL). The organic layer was dried (Na₂SO₄), concentrated in vacuo and the residue was purified by column chromatography on silica gel utilising (0-30%) EtOAc/hexane to provide methyl 3-bromo-5-fluoro-2,6-dianisate (0.348 g: 73% yield).

¹H NMR (300 MHZ, CDCl₃) δ −7.24 (dd, 1H), 3.86-3.81 (br, 6H), 3.74 (s, 3H).

¹⁹F NMR (300 MHZ, CDCl3) δ −132.05 ppm.

### Step 4: Methyl 5-fluoro-2,4-dimethoxy-3-toluate

A solution of methyl 3-bromo-5-fluoro-2,6-dianisate (100 mg, 0.34 mmol) in 1,4-dioxane (2 mL) in a microwave vial was degassed with argon for 20 min. Methylboronic acid (24.6 mg, 0.4 mmol), Pd(dppf)Cl₂ (12.5 mg, 0.017 mmol) and K₂CO₃ (70.8 mg, 0.51 mmol) were added then the reaction mixture was subjected to microwave radiation at 130° C. for 1 h. The mixture was extracted with EtOAc (30 mL) which was washed with water (2×10 mL) and brine (3×15 mL). The organic layer was dried (Na₂SO₄) concentrated, and the crude product was purified by column chromatography on silica gel eluting with (0-20% EtOAc/hexane) to afford methyl 5-fluoro-2,4-dimethoxy-3-toluate (48.0 mg: 61% yield).

¹H NMR (300 MHZ, CDCl₃) δ 7.24 (dd, 1H), 4.22 (s, 3H), 4.20 (d, 3H), 4.04 (s, 3H), 2.51 (s, 3H).

¹⁹F NMR (300 MHz, CDCl3) δ −135.25 ppm.

### Step 5: 3-fluoro-2,6-dimethoxy-5-methylbenzoic acid

To a solution of methyl 5-fluoro-2,4-dimethoxy-3-toluate (48.0 mg, 0.21 mmol) in CH₃OH (2 mL) in a microwave vial was added NaOH (9.26 mg, 0.23 mmol) pre-dissolved in water (1 mL). The reaction was subjected to microwave at 130° C. for 30 min, concentrated, redissolved in water (2 mL), and washed with CH₂Cl₂ (2×10 mL) to remove organic impurities. The aqueous layer was acidified with 1N HCl to PH ˜1, extracted with EtOAc (20 mL), dried (Na₂SO₄), filtered and concentrated to obtain 3-fluoro-2,6-dimethoxy-5-methylbenzoic acid (31.0 mg: 68% yield).

¹H NMR (300 MHZ, CD₃OD) δ 7.06 (dd, 1H), 3.90 (d, 3H), 3.78 (s, 3H), 2.25 (s, 3H).
¹⁹F NMR (300 MHZ, CDCl3) δ −137.37 ppm.

HPLC: Retention time 6.143 min, purity >98% at 280 nm.

### Example 2: Synthesis of 3-bromo-5-fluoro-2,6-dimethoxybenzoic acid (A-2)

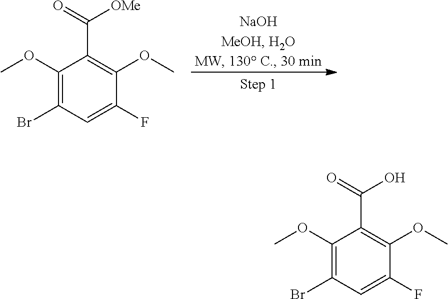

### Step 1: 3-Bromo-5-fluoro-2,6-dimethoxybenzoic acid

A solution of NaOH (15.07 mg, 0.38 mmol) in water (1 ml) was added to a solution of methyl 3-bromo-5-fluoro-2,6-dimethoxybenzoate (100.0 mg, 0.34 mmol) in CH₃OH (2 mL) and the reaction mixture was subjected to microwave conditions at 130° C. for 30 min. The mixture was evaporated to a residue, redissolved in water (2 ml), and washed with CH₂Cl₂ (2×10 mL). The aqueous layer was acidified with 1N HCl to PH ˜1 and extracted with EtOAc (20 mL). The solution was dried (Na₂SO₄), filtered and evaporated to obtain the title compound (76.0 mg, 80% yield).

1H NMR (300 MHZ, CD3OD) δ 7.52 (d, 1H), 3.95 (d, 3H), 3.87 (s, 3H).

19F NMR (300 MHZ, CD3OD) δ −133.95 ppm.

HPLC: Retention time 6.519 min, purity >98% at 280 nm.

### Example 3: 3,5-Dichloro-2,6-dimethoxybenzoic acid (A-3)

Compound is commercially available from e.g. Merck (PH006804).

### Example 4: Synthesis of 3-chloro-5,6-dimethyl-2-(trifluoromethoxy)benzoic acid (A-4)

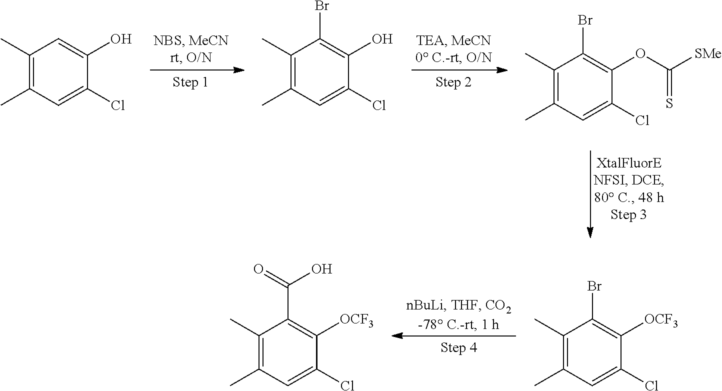

### Step 1: 2-Bromo-6-chloro-3,4-xylenol

To a solution of 2-chloro-4,5-xylenol (1.0 g, 6.41 mmol) in CH₃CN (25 mL) N-bromosuccinimide (1.14 g, 6.41 mmol) was introduced and the reaction mixture was stirred at ambient temperature for 18 h. It was then concentrated in vacuo, extracted with EtOAc (50 mL) and the solution washed with a saturated solution of NaHCO₃ (20 mL) and water (2×30 mL). The organic layer was dried (Na₂SO₄) and concentrated in vacuo. The residue was purified by column chromatography on silica gel eluting with (0-20%) EtOAc/hexane to afford 2-bromo-6-chloro-3,4-xylenol (1.45 g: 97% yield).

¹H NMR (300 MHZ, CDCl₃) δ 7.13 (s, 1H), 6.14 (s, 1H), 2.40 (s, 3H), 2.33 (s, 3H).

### Step 2: O-2-Bromo-6-chloro-3,4-dimethylphenyl S-methyl carbonodithioate

To a solution of 2-bromo-6-chloro-3,4-xylenol (0.50 g, 2.14 mmol) and triethylamine (0.327 mL, 2.35 mmol) in CH₃CN (10 mL) at 0° C., 3-methyl-1-(methylthiocarbonothioyl)-1H-imidazol-3-ium (0.37 g, 2.14 mmol) was added. The reaction was stirred at ambient temperature for 18 h. The reaction mixture was concentrated and extracted with EtOAc (50 mL), washed with saturated solution of NaHCO₃ (20 mL), and water (2×30 mL). The organic layer was dried over Na₂SO₄, concentrated in vacuo and the residue was purified by column chromatography on silica gel eluting with (0-20%) EtOAc/hexane to provide O-2-bromo-6-chloro-3,4-dimethylphenyl-S-methyl carbonodithioate (0.61 g: 87% yield).

1H NMR (300 MHZ, CDCl₃) δ 7.27 (s, 1H), 2.78 (s, 3H), 2.43 (s, 3H), 2.39 (s, 3H).

### Step 3: 3-Bromo-1-chloro-2-trifluoromethoxy-4,5-xylene

A mixture of O-2-bromo-6-chloro-3,4-dimethylphenyl-S-methyl-carbonodithioate (0.6 g, 1.85 mmol), XtalFluor-E (1.27 g, 5.56 mmol), and N-fluorobenzenesulphonimide (1.75 g, 5.56 mmol) in 1,2-dichloroethane (10 mL) was heated at 80° C. for 48 h. The reaction mixture was treated with a saturated solution of NaHCO₃ (10 mL), filtered through a celite pad and extracted with EtOAc (2×30 mL). The organic layer was washed with water (2×20 mL), dried over Na₂SO₄, concentrated in vacuo and the product purified by column chromatography on silica gel, eluting with (0-20%) EtOAc/hexane to provide 3-bromo-1-chloro-2-trifluoromethoxy-4,5-xylene (0.10 g: 17.9% yield).

¹H NMR (300 MHz, CDCl₃) δ 7.28 (s, 1H), 2.44 (s, 3H), 2.38 (s, 3H).

¹⁹F NMR (300 MHZ, CDCl3) δ −55.206 ppm.

### Step 4: 3-Chloro-5,6-dimethyl-2-(trifluoromethoxy)benzoic acid

n-BuLi (2.5 M in hexane) (0.073 mL, 0.182 mmol) was added to a solution of 3-bromo-1-chloro-2-trifluoromethoxy-4,5-xylene (50 mg, 0.165 mmol) in THF (4 mL) at −78° C. The reaction mixture was stirred at that temperature for 1 h and then CO₂ gas was bubbled through for 10 min and it was stirred for a further 1 h at −78° C. The reaction temperature was elevated to 0° C. and the pH was adjusted with 1M NaOH to ˜ 11. The mixture was extracted with CH2Cl2 (2×10 mL) to remove organic impurities; the water layer was acidified with 1N HCl to PH ˜1 and then extracted with EtOAc (20 mL). The solution was dried over Na₂SO₄, filtered and concentrated in vacuo. The crude product was purified by semi-prep HPLC utilising 30 to 100% acetonitrile/0.1% formic acid in water to afford 3-chloro-5,6-dimethyl-2-(trifluoromethoxy)benzoic acid (6.5 mg: 14.6% yield).

¹H NMR (300 MHZ, CD₃OD) δ 7.24 (s, 1H), 2.19 (s, 3H), 2.17 (s, 3H).

¹⁹F NMR (300 MHZ, CD₃OD) δ −57.560 ppm.

HPLC: Retention time 7.305 min, purity >98% at 280 nm.

### Example 5: Synthesis of 3-chloro-2-cyclopropoxy-5,6-dimethylbenzoic acid (A-5)

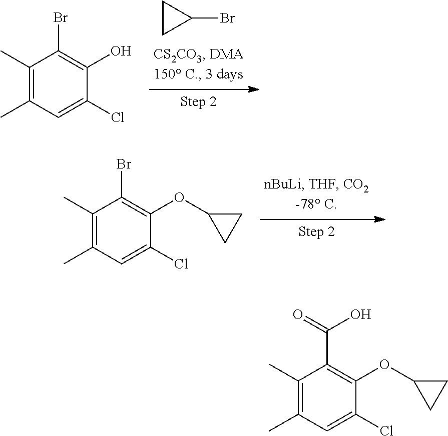

### Step 1: 3-Bromo-1-chloro-2-cyclopropoxy-4,5-dimethylbenzene

To a solution of 2-bromo-6-chloro-3,4-dimethylphenol (0.4 g, 1.7 mmol) in dimethyl acetamide (3 mL), cesium carbonate (1.66 g, 5.1 mmol, 3.0 eq.) was introduced, followed by bromocyclopropane (0.62 g, 5.1 mmol, 3.0 eq.). The reaction mixture was heated at 150° C. for 72 h, cooled, extracted with EtOAc (2×20 mL), then the combined extracts were washed with water (2×10 mL) and brine (10 mL). The organic layer was dried (Na₂SO₄) and concentrated in vacuo. The crude product was purified by column chromatography on silica gel eluting with (0-5%) EtOAc/hexane to provide 3-bromo-1-chloro-2-cyclopropoxy-4,5-dimethylbenzene (0.38 g, 81% yield).

¹H NMR (300 MHz, CDCl₃) δ 7.13 (s, 1H), 4.44-4.36 (m, 1H), 2.35 (s, 3H), 2.27 (s, 3H), 1.06-0.97 (m, 2H), 0.60-0.50 (m, 2H).

### Step 2: 3-Chloro-2-cyclopropoxy-5,6-dimethylbenzoic acid

To a solution of 3-bromo-1-chloro-2-cyclopropoxy-4,5-dimethylbenzene (0.2 g, 0.73 mmol) in THF (8 mL) at −78° C., n-BuLi (2.5 M in hexane) (0.32 mL, 0.8 mmol, 1.1 eq.) was added. The reaction mixture was stirred at that temperature for 30 min. After a further 30 min. CO₂ (g) was bubbled through the reaction mixture for 10 min. which was stirred for 1 h at −78° C. and slowly brought to 0° C. prior to quenching with 1M NaOH to pH ˜11. Washing with EtOAc (10 mL) was followed by separation of the water layer which was acidified with 1N HCl to PH ˜1. Extraction with EtOAc (2×10 mL) took place, and the combined extracts were washed with water (10 mL) and brine (10 mL). The organic layer was dried (Na₂SO₄), filtered and concentrated in vacuo to obtain 3-chloro-2-cyclopropoxy-5,6-dimethylbenzoic acid (0.083 g, 47% yield) as a white solid.

¹H NMR (300 MHz, CDCl₃) δ 10.55-9.50 (Br s, 1H), 7.25 (s, 1H), 4.41-4.33 (m, 1H), 2.26 (s, 3H), 2.25 (s, 3H), 0.98-0.90 (m, 2H), 0.62-0.53 (m, 2H).

ES-MS: m/z 239.3 (M−1).

HPLC: Retention time 7.262 min, purity >98% at 280 nm.

### Example 6: Synthesis of 2,5-dichloro-6-methoxy-3-methylbenzoic acid (A-6)

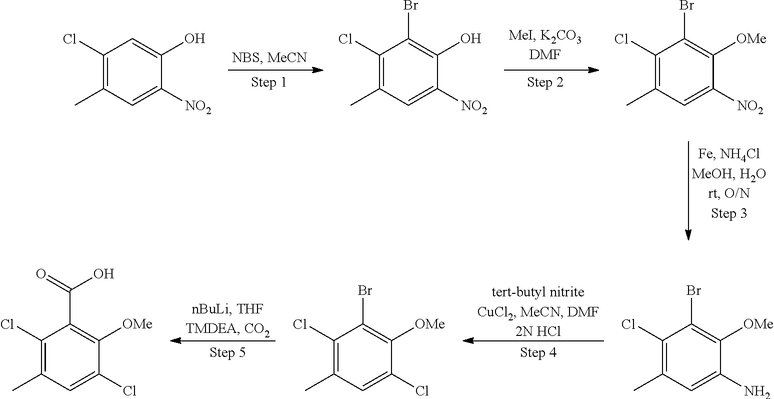

### Step 1: 3-Bromo-2-chloro-5-nitro-4-cresol

To a solution of 2-chloro-5-nitro-4-cresol (1.0 g, 5.35 mmol) in CH₃CN (10 mL) was added N-bromosuccinimide (952 mg, 5.35 mmol, 1.0 eq.). The reaction mixture was stirred at ambient temperature for 16 h. The reaction mixture was concentrated and extracted with EtOAc (2×50 mL) then the combined extracts were washed with water (3×50 mL) and brine (15 mL) before drying (Na₂SO₄). The crude product was obtained by evaporation in vacuo and purified by column chromatography on silica gel eluting with (0-5%) EtOAc/hexane to provide 3-bromo-2-chloro-5-nitro-4-cresol (1.50 g, 96%).

¹H NMR (300 MHZ, CDCl₃) δ 8.03 (br s, 1H), 8.02 (s, 1H), 2.42 (s, 3H) ppm.

### Step 2: 3-Bromo-2-chloro-4-methoxy-5-nitrotoluene

To a solution of 3-bromo-2-chloro-5-nitro-4-cresol (1.50 g, 5.64 mmol) in DMF (8 mL), K₂CO₃ (1.56 g, 11.28 mmol, 2 eq.) was added, followed by iodomethane (1.43 mL, 22.56 mmol, 4 eq.). The reaction mixture was stirred at ambient temperature for 18 h and the solvent was removed in vacuo and 2N HCl solution (20 mL) was added. Extraction with CH₂Cl₂ (3×30 mL) and drying (Na₂SO₄) provided the crude 3-bromo-2-chloro-4-methoxy-5-nitrotoluene after evaporation in vacuo as an oil (1.53 g, 96%).

¹H NMR (300 MHZ, CDCl₃) δ 7.69 (s, 1H), 3.97 (s, 3H), 2.44 (s, 3H).

### Step 3: 3-Bromo-4-chloro-2-methoxytolylamine

To a solution of 3-bromo-2-chloro-4-methoxy-5-nitrotoluene (2.0 g, 7.14 mmol, 1.0 eq.) in MeOH (100 mL) and water (100 mL), was added ammonium chloride solution (4.54 g, 85.68 mmol, 12 eq.), followed by Fe powder (2.4 g, 42.86 mmol). The reaction mixture was stirred at ambient temperature for 16 h and filtered through a plug of celite. The solvent was removed in vacuo, saturated NaHCO₃ solution (20 mL) was added and the mixture was extracted with CH₂Cl₂ (3×100 mL). The combined extracts were dried (Na₂SO₄) and concentrated to provide 3-bromo-4-chloro-2-methoxytolylamine as an oil 1.66 g (93% yield).

¹H NMR (300 MHZ, CDCl₃) δ 6.59 (s, 1H), 3.95 (br, 2H), 3.86 (s, 3H), 2.30 (s, 3H).

### Step 4: 3-Bromo-2,5-dichloro-4-methoxytoluene

CuCl₂ (1.66 g, 7.03 mmol) in CH₃CN was cooled to 0° C. and t-BuONO (1.253 mL, 10.55 mmol, 1.5 eq.) was added. After 2 min, 3-bromo-4-chloro-2-methoxytolylamine (1.66, 7.034 mL) in CH₃CN was added slowly and the reaction mixture was stirred for 30 min at 0° C. and then for 3 h. at ambient temperature. 2N HCl solution was added, the mixture was stirred for 30 min and the product extracted with EtOAc (3×100 mL) and the combined extracts were dried (Na₂SO₄) and concentrated in vacuo. Purification by silica gel chromatography eluting with (0-5%) EtOAc/hexane to afford 3-bromo-2,5-dichloro-4-methoxytoluene as a yellow solid (1.63 g, 86% yield).

¹H NMR (300 MHZ, CDCl₃) δ 7.26 (s, 1H), 3.90 (s, 3H), 2.40 (s, 3H).

### Step 5: 2,5-Dichloro-6-methoxy-3-methylbenzoic acid

To a solution of 3-bromo-2,5-dichloro-4-methoxytoluene (0.5 g, 1.865 mmol) in THF (10 mL) at −78° C. was added n-BuLi (2.5 M in hexane) (0.874 mL, 1.96 mmol, 1.05 eq.). The reaction mixture was stirred at that temperature for 50 min and CO₂ (g) was bubbled through it for 10 min. It was stirred for 1 h at −78° C., and slowly allowed to reach 0° C. before being quenched with 1M NaOH to reach PH ˜ 11. After washing with EtOAc (10 mL) the water layer was separated and acidified with 1N HCl to PH ˜1. Extraction was performed with EtOAc (3×50 mL) and the combined organic extracts were dried (Na₂SO₄) filtered then concentrated in vacuo to afford 2,5-dichloro-6-methoxy-3-methylbenzoic acid (0.3 g, 68%) as a white solid.

1H NMR (300 MHZ, CD3OD) δ 7.25 (s, 1H), 3.92 (s, 3H), 2.33 (s, 3H).

ES-MS: m/z 233.8 (M−1).

HPLC: Retention time 10.082 min, purity 96% at 280 nm.

### Example 7: Synthesis of 3-chloro-2,6-dimethoxy-5-methylbenzoic acid (A-7)

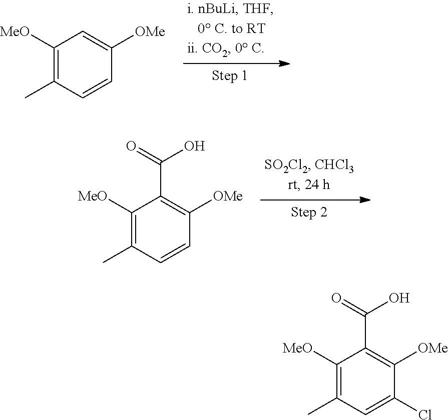

### Step 1: 2,6-Dimethoxy-3-methylbenzoic acid

To a solution of 2,4-dimethoxy-1-methylbenzene (2.00 g, 13.1 mmol) in THF (20 mL) at 0° C. was added n-BuLi (9.1 mL, 14.5 mmol, [1.6 M]), the reaction mixture was stirred at ambient temperature for 1 h. The reaction mixture was cooled to 0° C. and CO₂ gas was bubbled through it over a period of 45 min. The reaction mixture was quenched with water (25 mL) and washed with EtOAc (2×25 mL). The aqueous layer was acidified to pH ˜2 using 1.0 N HCl and extracted with EtOAc (2×25 mL). The combined organic extracts were dried (Na₂SO₄) and concentrated under reduced pressure to provide 2,6-dimethoxy-3-methylbenzoic acid (1.72 g, 67%).

¹H NMR (300 MHZ, CDCl₃) δ 7.27 (d, J=8.4 Hz, 1H), 6.72 (d, J=8.4 Hz, 1H), 3.93 (s, 3H), 3.92 (s, 3H), 2.32 (s, 3H).

ES-MS: 195.6 [M−H]

### Step 2: 3-Chloro-2,6-dimethoxy-5-methylbenzoic acid

To a solution of 2,6-dimethoxy-3-methylbenzoic acid (1.72 g, 8.77 mmol) in CHCl₃ (20 mL) at 0° C. ethanol (0.1 mL) was introduced, followed by sulphuryl chloride (1.41 mL, 17.5 mmol). The reaction mixture was stirred at ambient temperature for 24 h and the solvent was removed under reduced pressure. The residue was diluted with EtOAc (50 mL) and extracted with 1.0 N NaOH (2×50 mL). The combined aqueous extracts were acidified to PH ˜2 with 1.0 N HCl and extracted with EtOAc (2×50 mL). The combined organic extracts were dried (Na₂SO₄) and concentrated under reduced pressure. The crude product was subjected to column chromatography on silica gel eluting with 20% EtOAc/hexanes) to afford 3-chloro-2,6-dimethoxy-5-methylbenzoic acid (0.40 g, 20%).

¹H NMR (300 MHZ, CDCl₃) δ 7.28 (s, 1H), 3.93 (s, 3H), 3.84 (s, 3H), 2.25 (s, 3H).

ES-MS: 231.1 [M+H]

HPLC Retention Time: 9.60; purity >98% at 280 nm

### Example 8: Synthesis of 3-chloro-5,6-dimethyl-2-(methylsulphanyl)benzoic acid (A-8)

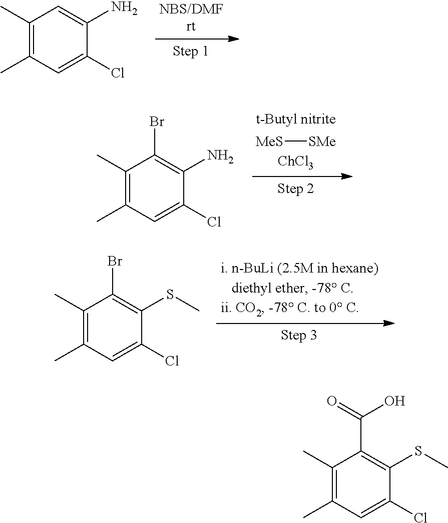

### Step 1: 2-Bromo-6-chloro-3,4-dimethylaniline

To a solution of 2-chloro-4,5-dimethylaniline (2.0 g, 12.9 mmol) in DMF (20 mL), N-bromosuccinimide (2.29 g, 12.9 mmol, 1.0 eq.) was added. The reaction mixture was stirred at ambient temperature for 16 h, concentrated in vacuo then water was added (20 mL). The mixture was extracted with EtOAc (2×40 mL) then the combined extracts were washed with water (3×20 mL) and brine (15 mL). The combined organic extracts were dried (Na₂SO₄) and concentrated in vacuo. The crude product was purified by column chromatography on silica gel eluting with (0-20%) EtOAc/hexane to provide 2-bromo-6-chloro-3,4-dimethylaniline (2.6 g, 86% yield).

¹H NMR (300 MHZ, CD₃OD) δ 7.04 (s, 1H), 2.32 (s, 3H), 2.22 (s, 3H).

### Step 2: (2-Bromo-6-chloro-3,4-dimethylphenyl)(methyl)sulphane

2-Bromo-6-chloro-3,4-dimethylaniline (0.8 g, 3.43 mmol, 1.0 eq.) in chloroform (6 mL) and tert-butyl nitrite (0.48 g, 4.64 mmol, 1.35 eq.) was simultaneously added into a solution of dimethyl disulphide (0.71 g, 7.55 mmol, 2.2 eq.) in chloroform (2 mL). The reaction mixture was stirred at ambient temperature for 30 min, extracted with chloroform (2×20 mL) and the combined extracts washed with water (10 mL), 1N HCl (10 mL) and brine (10 mL). The organic layer was dried over Na₂SO₄, filtered and concentrated in vacuo. The crude product was purified by column chromatography on silica gel using (0-10% EtOAc/hexane) to obtain (2-bromo-6-chloro-3,4-dimethylphenyl)(methyl)sulphane (0.52 g, 62% yield).

¹H NMR (300 MHZ, CDCl₃) δ 7.25 (s, 1H), 2.41 (s, 3H), 2.40 (s, 3H), 2.32 (s, 3H).

### Step 3: 3-Chloro-5,6-dimethyl-2-(methylsulphanyl)benzoic acid

To a solution of (2-bromo-6-chloro-3,4-dimethylphenyl)(methyl)sulphane (0.075 g, 0.307 mmol) in diethyl ether (3 mL) at −78° C., n-BuLi (2.5 M in hexane) (0.135 mL, 0.338 mmol, 1.1 eq.) was added. The mixture was stirred at that temperature for 1 h and after 1 h CO₂ (g) was bubbled through the reaction mixture for 10 min and stirred for 1 h at −78° C., slowly brought to 0° C. and quenched with 1M NaOH to PH ˜11. It was washed with EtOAc (5 mL) to remove non-acid impurities and water layer was separated then acidified with 1N HCl to PH ˜1 before extraction with EtOAc (2×10 mL) The combined extracts were washed with water (5 mL), brine (5 mL) and dried (Na₂SO₄), filtered and concentrated in vacuo to obtain 3-chloro-5,6-dimethyl-2-(methylsulphanyl)benzoic acid (0.026 g, 36% yield) as a white solid.

¹H NMR (300 MHZ, CDCl₃) δ 9.47-8.58 (br s, 1H), 7.34 (s, 1H), 2.40 (s, 3H), 2.28 (s, 3H), 2.27 (s, 3H).

ES-MS: m/z 229.34 (M−1).

HPLC: Retention time 10.497 min, purity >98% at 280 nm.

### Example 9: Synthesis of 3-chloro-2-(difluoromethoxy)-5,6-dimethylbenzoic acid (A-9)

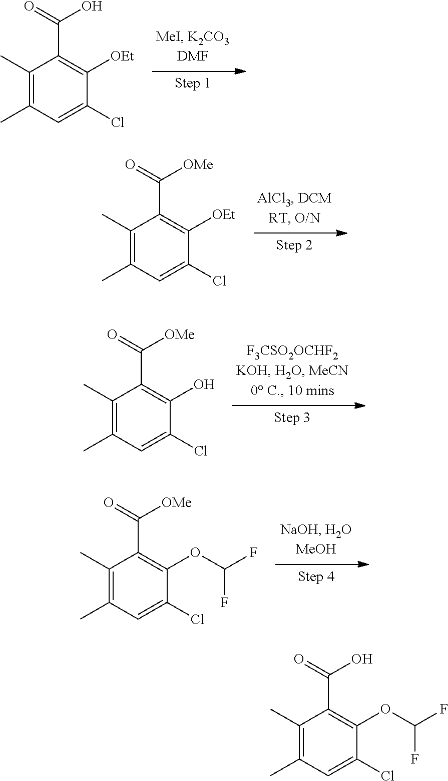

### Step 1: Methyl 3-chloro-2-ethoxy-5,6-xylenecarboxylate

To a solution of 3-chloro-2-ethoxy-5,6-xylenecarboxylic acid (600 mg, 2.63 mmol) in DMF (5 mL), K₂CO₃ (726 mg, 5.262 mmol, 2 eq.) was introduced, followed by iodomethane (0.5 mL, 7.9 mmol, 3 eq.) and the reaction mixture was stirred at ambient temperature for 18 h. The solvent was removed in vacuo and the residue treated with 2N HCl solution (20 mL) before extraction with CH₂Cl₂ (3×50 mL). The combined extracts were dried (Na₂SO₄) and concentrated to provide methyl 3-chloro-2-ethoxy-5,6-xylenecarboxylate a greenish oil (620 mg, 97%).

¹H NMR (300 MHZ, CDCl₃) δ 7.25 (s, 1H), 4.15 (q, 2H), 3.94 (s, 3H), 2.30 (s, 3H), 2.35 (s, 3H), 1.49 (t, 3H).

### Step 2: Methyl 3-chloro-2-hydroxy-5,6-xylenecarboxylate

To a solution of methyl 3-chloro-2-ethoxy-5,6-xylenecarboxylate (370 mg, 1.525 mmol, 1.0 eq.) in DCM (15 mL) at 0° C., AlCl₃ (811 mg, 6.1 mmol, 4.0 eq.) was added. The reaction mixture was stirred at ambient temperature for 18 h. The reaction was quenched with 2N HCl solution (10 mL) before extraction with CH₂Cl₂ (3×50 mL). The combined extracts were dried (Na₂SO₄) and concentrated to provide methyl 3-chloro-2-hydroxy-5,6-xylenecarboxylate as a light yellow solid (310 mg, 95%).

¹H NMR (300 MHZ, CDCl₃) δ 7.26 (s, 1H), 3.98 (s, 3H), 2.37 (s, 3H), 2.30 (s, 3H).

### Step 3: Methyl 3-chloro-2-difluoromethoxy-5,6-xylenecarboxylate

To a solution of methyl 3-chloro-2-hydroxy-5,6-xylenecarboxylate (230 mg, 1.075 mmol, 1.0 eq.) in CH₃CN (3 mL) at 0° C. was added 6N KOH solution (2.5 mL), followed by dropwise addition of difluoromethoxysulphonyltrifluoromethane (0.344 mL, 1.72 mmol, 1.6 eq.). The reaction mixture was stirred at 0° C. for 10 min. The reaction mixture was quenched with water (20 mL) and extracted with CH₂Cl₂ (3×50 mL), dried (Na₂SO₄) then concentrated in vacuo to an oil which was purified by silica gel column chromatography eluting with (0-5%) EtOAc/hexane to provide methyl 3-chloro-2-difluoromethoxy-5,6-xylenecarboxylatecolourless oil (210 mg, 80%).

1H NMR (300 MHZ, CDCl₃) δ 7.26 (s, 1H), 6.46 (t, 1H), 3.91 (s, 3H), 2.23 (s, 3H), 2.16 (s, 3H) ppm

¹⁹F NMR (300 MHZ, CDCl₃) δ −80.52 ppm.

### Step 4: 3-chloro-2-(difluoromethoxy)-5,6-dimethylbenzoic acid

To a solution of methyl 3-chloro-2-difluoromethoxy-5,6-xylenecarboxylate (85 mg, 0.332 mmol, 1.0 eq.) in MeOH (1 mL) and water (2 mL) 1N solution of NaOH (0.35 mL, 0.354 mmol 1.1 eq.) was added. The mixture was heated in a microwave reactor at 150° C. for 50 min. The solvent was removed in vacuo and the residue was dissolved in water (15 mL), washed with CH₂Cl₂ (3×10 mL), neutralized with 2N HCl then extracted with CH₂Cl₂ (3×20 mL). The combined extracts provided 3-chloro-2-(difluoromethoxy)-5,6-dimethylbenzoic acid as a yellow solid (75 mg, 98%).

¹H NMR (300 MHZ, CD₃OD) δ 7.04 (s, 1H), 6.46 (t, 1H), 2.13 (s, 6H) ppm

¹⁹F NMR (300 MHZ, CD₃OD) δ −81.64 ppm.

ES-MS: m/z 249.2 (M−1).

HPLC: Retention time 12.18 min, purity 98% at 280 nm.

### Example 10: Synthesis of 3-chloro-2-(fluoromethoxy)-5,6-dimethylbenzoic acid (A-10)

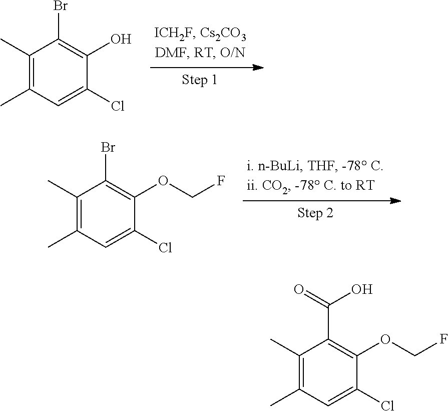

### Step 1: 3-Bromo-1-chloro-2-fluoromethoxy-4,5-xylene

To a solution of 2-bromo-6-chloro-3,4-xylenol (205 mg, 0.872 mmol) in DMF (3 mL), Cs₂CO₃ (567 mg, 1.74 mmol, 2 eq.) was added fluoroiodomethane (418 mg, 2.613 mmol 3.0 eq.) by slow addition. The reaction mixture was stirred at ambient temperature for 18 h, concentrated in vacuo and saturated aqueous NaHCO₃ solution (25 mL) was added and extracted with CH₂Cl₂ (2×40 mL). The combined extracts were dried (Na₂SO₄), filtered and concentrated in vacuo to provide a white solid (200 mg, 86%).

¹H NMR (300 MHZ, CDCl₃) δ 7.26 (s, 1H), 5.72 (s, 1H), 5.54 (s, 1H), 2.30 (s, 3H), 2.25 (s, 3H) ppm.

¹⁹F NMR (300 MHZ, CDCl₃) δ −149.23 ppm

### Step 2: 3-Chloro-2-(fluoromethoxy)-5,6-dimethylbenzoic acid

To a solution of 3-bromo-1-chloro-2-fluoromethoxy-4,5-xylene (0.2 g, 0.75 mmol, 1.0 eq.) in THF (5 mL) at −78° C. a solution of 2.5 M n-BuLi in hexane (0.3 mL, 0.75 mmol, 1.0 eq.) was added. The reaction mixture was stirred at this temperature for 45 min. After 50 min. CO₂ (g) was bubbled for 10 min., and the reaction mixture stirred for 1 h at −78° C. and was slowly brought to ambient temperature before quenching with 1M NaOH to pH ˜11. The mixture was washed with CH₂Cl₂ (20 mL) to remove impurities then the water layer was separated and acidified with 1N HCl to PH ˜1 followed by extraction with EtOAc (2×10 mL). The organic layer was dried (Na₂SO₄), filtered, and evaporated to obtain 3-chloro-2-(fluoromethoxy)-5,6-dimethylbenzoic acid (80 mg, 46%) as a white solid.

¹H NMR (300 MHZ, CD₃OD) δ 7.35 (s, 1H), 5.72 (s, 1H), 5.54 (s, 1H), 2.30 (s, 3H), 2.25 (s, 3H).

¹⁹F NMR (300 MHZ, CD₃OD) δ −149.54

ES-MS: m/z 231.4 (M−1).

HPLC: Retention time 9.43 min, purity 89% at 280 nm.

### Example 11: Synthesis of 3-fluoro-2-methoxy-5,6-dimethylbenzoic acid (A-11)

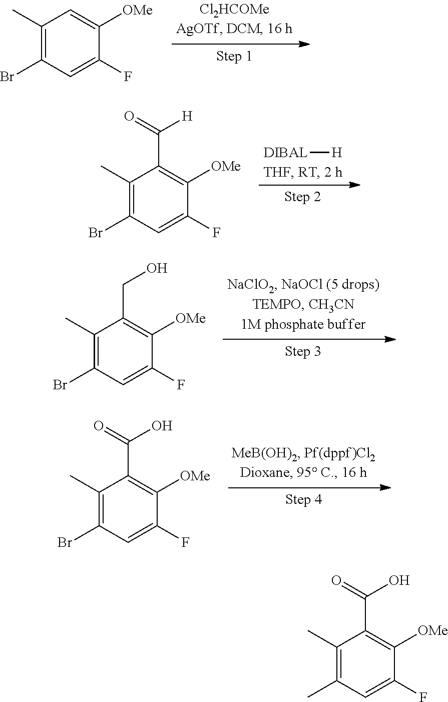

### Step 1: 3-Bromo-5-fluoro-6-methoxy-2-methylbenzaldehyde

To a suspension of 1-bromo-5-fluoro-4-methoxy-2-methylbenzene (607 mg, 2.76 mmol) and AgOTf (2.12 g, 8.28 mmol) in dry CH₂Cl₂ (4 mL) a solution of Cl₂CHOMe (0.75 mL, 8.28 mmol) in dry CH₂Cl₂ (2 mL) was added at −78° C. under an argon atmosphere. After stirring at ambient temperature for 16 h, the reaction mixture was quenched with saturated aqueous NaHCO₃. Following further stirring at ambient temperature for 30 min, the reaction mixture was filtered through a pad of celite, the organic layer was separated and the aqueous layer was extracted with CH₂Cl₂ (2×40 mL). The combined organic layers were washed with brine, dried (Na₂SO₄) and filtered. The filtrate was concentrated in vacuo and the resultant residue was purified by flash column chromatography on silica gel eluting with (0-10%) EtOAc/hexane to afford 3-bromo-5-fluoro-6-methoxy-2-methylbenzaldehyde (479 mg, 70% yield) as off-white solid.

¹H NMR (300 MHZ, CDCl₃) δ 10.51 (s, 1H); 7.59 (d, 1H); 4.09 (d, 3H); 2.63 (s, 3H); ES-MS: 248 [M+1].

¹⁹F NMR (300 MHZ, CDCl₃) δ −131.16.

### Step 2: (3-Bromo-5-fluoro-6-methoxy-2-methylphenyl) methanol

To a solution of 3-bromo-5-fluoro-6-methoxy-2-methylbenzaldehyde (150 mg, 0.60 mmol) in THF (10 mL) at 0° C. DIBAL-H (1M in toluene, 0.91 mL, 0.91 mmol) was added dropwise over 20 min. The solution was stirred for a further 15 min at 0° C. The solution was allowed to warm to ambient temperature and stirred for 2 h. The reaction was quenched with saturated aqueous sodium potassium tartrate and extracted with EtOAc (2×50 mL). The combined organic layers were washed with brine (50 mL) dried over (Na₂SO₄), filtered and evaporated. The crude product was purified by chromatography on silica gel (0-10%) EtOAc/hexane to afford (3-bromo-5-fluoro-6-methoxy-2-methylphenyl) methanol (112 mg, 98% yield) as a colourless oil.

¹H NMR (300 MHZ, CDCl₃) δ 7.24 (d, 1H); 4.69 (s, 2H); 3.88 (d, 3H); 2.37 (s, 3H); ES-MS: 250 [M+1].

¹⁹F NMR (300 MHZ, CDCl₃) δ −131.16.

### Step 3: 3-Bromo-5-fluoro-6-methoxy-2-methylbenzoic acid

To a stirred solution of (3-bromo-5-fluoro-6-methoxy-2-methylphenyl) methanol (155 mg, 0.84 mmol), sodium chlorite (189 mg, 2.1 mmol) in acetonitrile (7 mL) and 1M sodium phosphate buffer (pH˜6, 7 mL) sodium hypochlorite solution (5 drops, 4-4.99M) was added, followed by TEMPO (6.5 mg, 0.042 mmol). The reaction mixture was stirred at ambient temperature for 2 h. Further sodium hypochlorite (4 drops, 4-4.99M solution) and TEMPO (6.5 mg, 0.042 mmol) were introduced; the addition of sodium hypochlorite and TEMPO was repeated three more times at 8 h intervals. After completion of the reaction, it was cooled to 0° C. and 1M NaOH solution was added to adjust pH ˜13 and extracted with CH₂Cl₂ (40 mL). The water layer was separated and cooled again to 0° C., acidified with 1M HCl to PH ˜1, extracted with EtOAc (2×50 mL), washed with water (2×10 mL), brine (10 mL), dried (Na₂SO₄), filtered and concentrated to provide 3-bromo-5-fluoro-6-methoxy-2-methylbenzoic acid as a white solid (120 mg, 96%).

¹H NMR (300 MHZ, CDCl₃) δ 10.94-10.38 (br, 1H), 7.28 (d, 1H); 3.86 (d, 3H); 2.27 (s, 3H); ES-MS: 363 [M−1].

¹⁹F NMR (300 MHZ, CDCl₃) δ −130.38.

### Step 4: 3-Fluoro-2-methoxy-5,6-dimethylbenzoic acid

A mixture of 3-bromo-5-fluoro-6-methoxy-2-methylbenzoic acid (50 mg, 0.19 mmol), Pd(dppf)Cl₂ (6.93 mg, 0.0095 mmol), methyl boronic acid (22.6 mg, 0.38 mmol) and potassium carbonate (78.5 mg, 0.57 mmol) in 1,4-dioxane (0.85 mL) and water (0.85 mL) was evacuated and purged with argon three times and the mixture was heated at 90° C. for 12 h then allowed to cool to ambient temperature. Volatiles were removed in vacuo and the crude product was purified by semi-prep HPLC (10 to100% acetonitrile/0.1% formic acid in water) to afford 3-fluoro-2-methoxy-5,6-dimethylbenzoic acid as an off-white solid (25 mg, 96.1%).

¹H NMR (300 MHZ, CDCl₃) δ 10.97-10.15 (br, 1H), 6.97 (d, 1H); 3.96 (d, 3H); 2.24 (s, 3H); 2.243 (s, 3H); ES-MS: 197 [M−1].

¹⁹F NMR (300 MHZ, CDCl₃) δ −133.88.

HPLC Retention Time: 8.865 min, purity >98% at 280 nm.

### Example 12: Synthesis of 3-chloro-2-ethoxy-5,6-dimethylbenzoic acid (A-12)

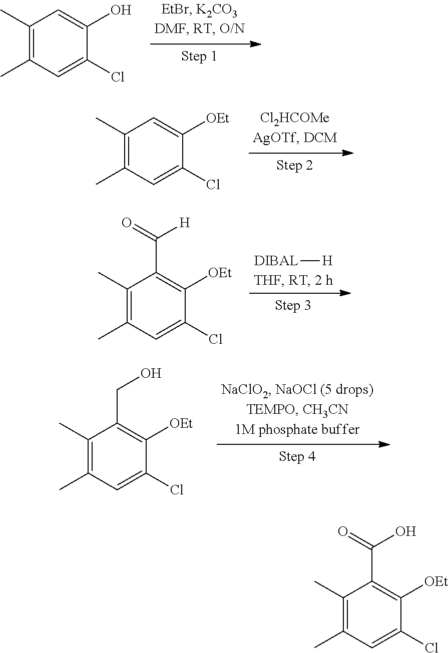

### Step 1:1-Chloro-2-ethoxy-4,5-xylene

To a solution of 2-chloro-4,5-xylenol (1.0 g, 6.39 mmol, 1.0 eq.) in DMF (5 mL), K₂CO₃ (1.762 g, 12.77 mmol, 2 eq.) was introduced followed by slow addition of ethyl bromide (1.9 mL, 25.54 mmol, 4 eq.). The reaction mixture was stirred at ambient temperature for 18 h. The solvent was removed in vacuo, saturated NaHCO₃ solution (15 mL) was added to the residue and the mixture extracted with CH₂Cl₂ (3×20 mL) and the combined extracts dried (Na₂SO₄) and concentrated to afford 1-chloro-2-ethoxy-4,5-xylene as a white solid (1.1 g, 93%).

¹H NMR (300 MHZ, CDCl₃) δ 7.27 (s, 1H), 6.71 (s, 1H), 4.10 (q, 2H), 2.35 (s, 3H), 2.30 (s, 3H), 1.41 (t, 3H).

### Step 2: 3-Chloro-2-ethoxy-5,6-xylenecarbaldehyde

To a suspension of 1-chloro-2-ethoxy-4,5-xylene (1.13 g, 6.12 mmol) and AgOTf (4.72 g, 18.36 mmol, 3.0 eq.) in dry CH₂Cl₂ (12 mL) a solution of Cl₂CHOMe (1.75 mL, 18.36 mmol, 3.0 eq.) in dry CH₂Cl₂ (2 mL) was added at −78° C. under an argon atmosphere. After being stirred at ambient temperature for 16 h, the reaction mixture was quenched with saturated aqueous NaHCO₃ (30 mL). After a further 30 min of stirring at ambient temperature the reaction mixture was filtered through a pad of celite. The organic layer was separated, and the aqueous layer was extracted with CH₂Cl₂ (2×30 mL). The combined organic layers were washed with brine, dried (Na₂SO₄) and filtered. The filtrate was concentrated in vacuo and the resultant residue was purified by flash chromatography on silica gel (0-10%) EtOAc/hexane to afford 3-chloro-2-ethoxy-5,6-xylenecarbaldehyde (970 mg, 86%) as an off-white solid.

¹H NMR (300 MHZ, CDCl₃) δ 10.40 (s, 1H), 7.30 (s, 1H), 4.11 (q, 2H), 2.40 (s, 3H), 2.33 (s, 3H), 1.42 (t, 3H).

### Step 3: (3-Chloro-2-ethoxy-5,6-xylyl) methanol

To a solution of 3-chloro-2-ethoxy-5,6-xylenecarbaldehyde (950 mg, 4.47 mmol) in THF (10 mL) at 0° C., DIBAL-H (1M in toluene, 13.4 mL, 13.40 mmol, 3 eq.) was added dropwise over 5 min. The solution was stirred for a further 15 min at 0° C., allowed to warm to ambient temperature and stirred for a further 16 h. The reaction mixture was quenched with saturated aqueous sodium potassium tartrate (20 mL), stirred for 20 min, filtered through pad of celite and extracted with EtOAc (2×50 mL). The combined organic extracts were washed with brine (50 mL) dried (Na₂SO₄) and evaporated to provide (3-chloro-2-ethoxy-5,6-xylyl) methanol (861 mg, 90%) as a yellow oil.

¹H NMR (300 MHZ, CDCl₃) δ 7.18 (s, 1H), 4.80 (d, 2H), 4.04 (q, 2H), 2.27 (s, 3H), 2.31 (s, 3H), 1.46 (t, 3H).

### Step 4: 3-Chloro-2-ethoxy-5,6-dimethylbenzoic acid

To a stirred solution of (3-chloro-2-ethoxy-5,6-xylyl) methanol (766 mg, 3.58 mmol), sodium chlorite (809 mg, 8.92 mmol, 2.5 eq.) in acetonitrile (14 mL) and 1M sodium phosphate buffer (pH˜6, 14 mL) was added sodium hypochlorite (0.7 mL, 4-4.99M solution) followed by TEMPO (112 mg, 0.716 mmol, 0.2 eq.). The reaction mixture was stirred at ambient temperature for 16 h. After completion of the reaction the mixture was cooled to 0° C. and 1M NaOH solution was added adjusting the pH to ˜13 and the solution was washed with CH₂Cl₂ (100 mL). The water layer was separated, cooled to 0° C., acidified with 1M HCl to PH ˜1, extracted with EtOAc (3×50 mL), and the combined extracts were washed with water (2×20 mL), brine (10 mL), dried (Na₂SO₄) and evaporated to provide 3-chloro-2-ethoxy-5,6-dimethylbenzoic acid as a white solid (680 mg, 83% yield).

¹H NMR (300 MHZ, CDCl₃) δ 7.30 (s, 1H), 4.21 (q, 2H), 2.30 (s, 3H), 2.33 (s, 3H), 1.42 (t, 3H).

ES-MS: m/z 227.5 (M−1).

HPLC: Retention time 10.384 min, purity 97% at 254 nm.

### Example 13: Synthesis of 3-bromo-2-methoxy-5,6-dimethylbenzoic acid (A-13)

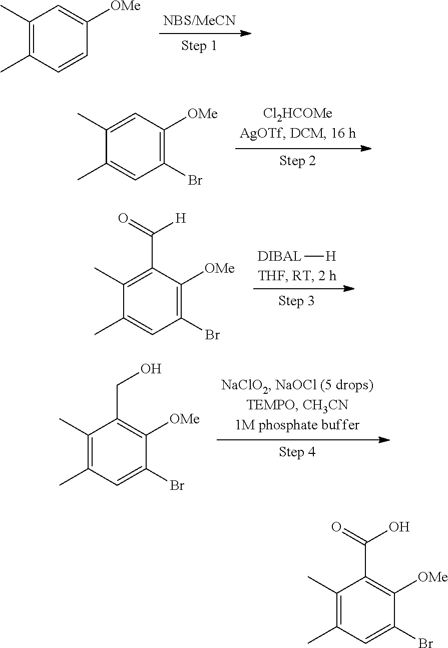

### Step 1:1-Bromo-2-methoxy-4,5-dimethylbenzene

A mixture of 4-methoxy-1,2-dimethylbenzene (1.4 g, 10.28 mmol) and N-bromosuccinimide (1.99 g, 11.2 mmol) in acetonitrile (25 mL) was stirred at ambient temperature for 18 h. The reaction mixture was quenched with water (40 mL) and extracted with EtOAc (2×100 mL). The combined organic extracts were washed with saturated brine (50 mL), dried (Na₂SO₄), filtered and evaporated. The crude product was purified by column chromatography on silica gel (0-10%) EtOAc/hexane to afford 1-bromo-2-methoxy-4,5-dimethylbenzene (1.9 g, 86% yield) as an oil.

¹H NMR (300 MHZ, CDCl₃) δ 7.17 (s, 1H); 6.78 (s, 1H); 3.92 (s, 3H); 2.29 (s, 3H); 2.23 (s, 3H). ES-MS: 171 [M+1].

### Step 2: 3-Bromo-2-methoxy-5,6-dimethylbenzaldehyde

To a suspension of 1-bromo-2-methoxy-4,5-dimethylbenzene (1 g, 4.65 mmol) and AgOTf (3.58 g, 13.95 mmol) in dry CH₂Cl₂ (8 mL), a solution of Cl₂CHOMe (1.26 mL, 13.95 mmol) in dry CH₂Cl₂ (5 mL) was added at −78° C. under an argon atmosphere. The rection mixture was stirred and gradually allowed to reach ambient temperature over 16 h, the reaction mixture was quenched with saturated aqueous NaHCO₃ then stirring was continued for 30 min. The reaction mixture was filtered through a pad of celite, the organic layer separated and the aqueous layer extracted with CH₂Cl₂ (2×30 mL). The combined organic extracts were washed with brine, dried (Na₂SO₄) and filtered. The filtrate was concentrated in vacuo and the resultant residue was purified by flash column chromatography on silica gel (0-10%) EtOAc/hexane to afford 3-bromo-2-methoxy-5,6-dimethylbenzaldehyde (553 mg, 49% yield) as a solid.

¹H NMR (300 MHZ, CDCl₃) δ 10.55 (s, 1H); 7.60 (s, 1H); 3.95 (s, 3H); 2.48 (s, 3H); 2.32 (s, 3H). ES-MS: 245 [M+1].

### Step 3: 3-Bromo-2-methoxy-5,6-dimethylphenyl) methanol

To a solution of 3-bromo-2-methoxy-5,6-dimethylbenzaldehyde (366 mg, 1.51 mmol) in THF (15 mL) at 0° C., DIBAL-H (1M in toluene, 2.26 mL, 2.26 mmol) was introduced dropwise over 20 min. The solution was stirred for 15 min at 0° C. and was allowed to warm to ambient temperature and stirred for a further 2 h. The reaction was quenched with saturated aqueous sodium potassium tartrate solution (10 mL) and extracted with EtOAc (2×50 mL). The combined organic layers were washed with brine (50 mL), dried (Na₂SO₄) filtered and evaporated. The crude residue was purified by chromatography on silica gel (0-10%) EtOAc/hexane to afford (3-bromo-2-methoxy-5,6-dimethylphenyl) methanol (350 mg, 95% yield) as a colourless oil.

¹H NMR (300 MHZ, CDCl₃) δ 7.37 (s, 1H); 4.83 (s, 2H); 3.92 (s, 3H); 2.32 (s, 3H); 2.28 (s, 3H). ES-MS: 246 [M+1].

### Step 4: 3-Bromo-2-methoxy-5,6-dimethylbenzoic acid

To a stirred solution of (3-bromo-2-methoxy-5,6-dimethylphenyl) methanol (350 mg, 1.43 mmol), sodium chlorite (323 mg, 3.58 mmol) in acetonitrile (12 mL) and 1M sodium phosphate buffer (pH˜6, 12 mL) was added sodium hypochlorite (5 drops, 4-4.99M solution) followed by TEMPO (11.1 mg, 0.0715 mmol). The reaction mixture was stirred at ambient temperature for 2 h. Further sodium hypochlorite (4 drops, 4-4.99M solution) and TEMPO (11.1 mg, 0.0715 mmol) were added and addition of this sodium hypochlorite/TEMPO reagent took place three more times at 8 h intervals. After completion of the reaction, the mixture was cooled to 0° C. and 1M NaOH solution was added to adjust the pH to ˜13 before being extracted with CH₂Cl₂ (40 mL). The water layer was separated and cooled again to 0° C., acidified with 1M HCl to PH ˜1, extracted with EtOAc (2×50 mL). The combined extracts were washed with water (2×10 mL), brine (10 mL), dried (Na₂SO₄). filtered and concentrated to provide 3-bromo-2-methoxy-5,6-dimethylbenzoic acid as a white solid (455 mg, 89%).

¹H NMR (300 MHZ, CDCl₃) δ 11.91-11.57 (br, 1H), 7.44 (s, 1H); 3.99 (s, 3H); 2.29 (s, 3H); 2.28 (s, 3H).

ES-MS: 258 [M−1].

HPLC Retention Time: 9.554 min, >98% purity at 280 nm.

### Example 14: Synthesis of 3-chloro-2-methoxy-5,6-dimethylbenzoic acid (A-14)

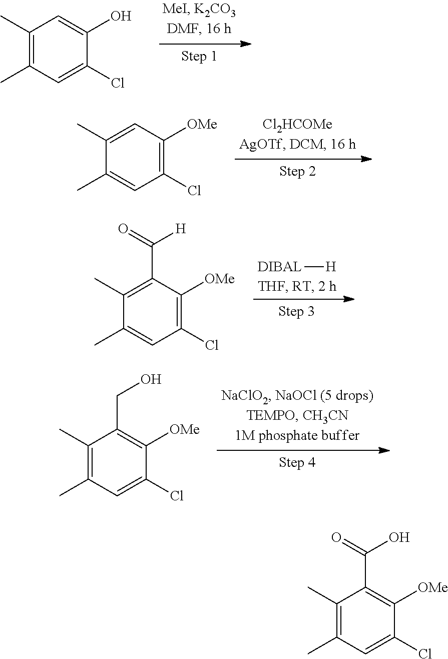

### Step 1:1-Chloro-2-methoxy-4,5-dimethylbenzene

To a solution of 2-chloro-4,5-dimethylphenol (3.07 g, 19.6 mmol) and K₂CO₃ (5.41 g, 39.2 mmol) in DMF (25 mL) methyl Iodide (2.44 mL, 39.2 mmol) was added and the resulting solution was stirred at ambient temperature for 18 h. The reaction mixture was quenched with water (40 mL) and extracted with EtOAc (2×100 mL). The combined organic extracts were washed with brine (50 mL), dried (Na₂SO₄), filtered and evaporated. The crude product was purified by column chromatography on silica gel (0-10%) EtOAc/hexane to afford 1-chloro-2-methoxy-4,5-dimethylbenzene (3.31 g, 98% yield) as a colourless oil.

¹H NMR (300 MHZ, CDCl₃) δ 7.17 (s, 1H); 6.78 (s, 1H); 3.92 (s, 3H); 2.29 (s, 3H); 2.23 (s, 3H). ES-MS: 171 [M+1].

### Step 2: 3-Chloro-2-methoxy-5,6-dimethylbenzaldehyde

To a suspension of 1-chloro-2-methoxy-4,5-dimethylbenzene (1.4 g, 8.2 mmol) and AgOTf (6.3 g, 24.6 mmol) in dry CH₂Cl₂ (12 mL), a solution of Cl₂CHOMe (2.22 mL, 24.6 mmol) in dry CH₂Cl₂ (6 mL) was added at −78° C. under an argon atmosphere. After stirring at ambient temperature for 16 h, the reaction mixture was quenched with saturated aqueous NaHCO₃. Following further stirring at ambient temperature for 30 min, the reaction mixture was filtered through a pad of celite. The organic layer was separated and the aqueous layer extracted with CH₂Cl₂ (2×50 mL). The combined organic layers were washed with brine (50 mL), dried (Na₂SO₄) and filtered. The filtrate was concentrated and the resultant residue was purified by flash column chromatography on silica gel (0-10%) to afford 3-chloro-2-methoxy-5,6-dimethylbenzaldehyde (1.2 g, 86% yield) as a white solid.

¹H NMR (300 MHZ, CDCl₃) δ 10.54 (s, 1H); 7.41 (s, 1H); 3.95 (s, 3H); 2.48 (s, 3H); 2.28 (s, 3H). ES-MS: 199 [M+1].

### Step 3: (3-Chloro-2-methoxy-5,6-dimethylphenyl) methanol

To a solution of 3-chloro-2-methoxy-5,6-dimethylbenzaldehyde (565 mg, 2.84 mmol) in THF (17 mL) at 0° C., DIBAL-H (1M in toluene, 4.26 mL, 4.26 mmol) was added dropwise over 20 min. The solution was stirred for 15 min at 0° C. and allowed to warm to ambient temperature and stirred for a further 2 h. The reaction was quenched with saturated aqueous sodium potassium tartrate and extracted with EtOAc (2×50 mL). The combined organic extracts were washed with brine (50 mL), dried (Na₂SO₄), filtered and evaporated. The crude residue was purified by chromatography on silica gel (0-10%) EtOAc/hexane to afford (3-chloro-2-methoxy-5,6-dimethylphenyl) methanol (475 mg, 83% yield) as a colourless oil.

¹H NMR (300 MHZ, CDCl₃) δ 7.23 (s, 1H); 4.84 (s, 2H); 3.92 (s, 3H); 2.34 (s, 3H); 2.29 (s, 3H).

ES-MS: 201 [M+1].

### Step 4: 3-Chloro-2-methoxy-5,6-dimethylbenzoic acid

To a stirred solution of (3-chloro-2-methoxy-5,6-dimethylphenyl) methanol (475 mg, 2.37 mmol), sodium chlorite (535 mg, 5.93 mmol) in acetonitrile (7 mL) and 1M sodium phosphate buffer (pH˜6, 7 mL), sodium hypochlorite (5 drops, 4-4.99M solution) were added followed by TEMPO (18.4 mg, 0.118 mmol). The reaction mixture was stirred at ambient temperature for 2 h. Further sodium hypochlorite (4 drops, 4-4.99M solution) plus TEMPO (18.4 mg, 0.118 mmol) were added and addition of this sodium hypochlorite/TEMPO reagent took place three more times at 8 h intervals. After completion, the reaction mixture was cooled to 0° C. and 1M NaOH solution was added to adjust pH ˜13 and extracted with CH₂Cl₂ (40 mL). The water layer was separated and cooled again to 0° C., acidified with 1M HCl to PH ˜1, extracted with EtOAc (2×50 mL), washed with water (2×10 mL), brine (10 mL), dried (Na₂SO₄), filtered and concentrated in vacuo to provide 3-chloro-2-methoxy-5,6-dimethylbenzoic acid as a white solid (455 mg, 89%).

¹H NMR (300 MHZ, CDCl₃) δ 10.94-10.38 (br, 1H), 7.32 (s, 1H); 3.99 (s, 3H); 2.33 (s, 3H); 2.31 (s, 3H).

ES-MS: 215 [M−1].

HPLC Retention Time: 9.683 min, purity >98% at 280 nm.

### Example 15: Electrophysiological Measurement of Compound Inhibition of CIC-1 in Rat Muscle

The investigatory goal of these experiments was to evaluate whether compounds inhibit CIC-1 channels in native tissue of rat skeletal muscle fibres. Apparent CIC-1 affinity was reported by the concentration of compound at which 50% of the compound's full inhibition of CIC-1 was observed (EC₅₀).

Experimentally, Gₘ was measured in individual fibres of whole rat soleus muscles using a three micro-electrodes technique described in this example and in full detail elsewhere (Riisager et al., Determination of cable parameters in skeletal muscle fibres during repetitive firing of action potentials. Journal of Physiology, 2014, 592, 4417-4429). Briefly, intact rat soleus muscles were dissected out from 12-14 week old Wistar rats and placed in an experimental chamber that was perfused with a standard Krebs Ringer solution containing 122 mM NaCl, 25 mM NaHCO₃, 2.8 mM KCl, 1.2 mM KH₂PO₄, 1.2 mM MgSO₄, 1.3 mM CaCl₂), 5.0 mM D-glucose. During experiments, the solution was kept at approx. 30° C. and continuously equilibrated with a mixture of 95% O₂ and 5% CO₂, PH ˜7.4. The experimental chamber was placed in Nikon upright microscope that was used to visualize individual muscle fibres and the three electrodes (glass pipettes filled with 2 M potassium citrate). For Gₘ measurements, the electrodes were inserted into the same fibre with known inter-electrode distances of 0.35-0.5 mm (V1-V2, X1) and 1.1-1.5 mm (V1-V3, X3). The membrane potential of the impaled muscle fibre was recorded by all electrodes. Two of the electrodes were furthermore used to inject 50 ms current pulses of −30 nA. Given the positions of the electrodes, three different inter-electrode distances could be identified (X1-X2, X1-X3, X2-X3) and hence the membrane potential responses to the current injections could be obtained at three distances from the point of current injection. The steady state voltage deflection at each distance was divided by the magnitude of current injected (−30 nA) and the resulting transfer resistances were plotted against inter-electrode distance and the data was fitted to a mono-exponential function from which Gₘ could be calculated using linear cable theory.

To establish a dose response relationship, Gₘ was first determined in 10 muscle fibres in the absence of compound and then at four increasing compound concentrations with Gₘ determinations in 5-10 fibres at each concentration. The average Gₘ values at each concentration were plotted against compound concentration and the data was fitted to sigmoidal function to obtain an EC₅₀ value. Table 1 shows the EC₅₀ values for a range of compounds with n values referring to number of experiments that each reflect recordings from around 50 fibres.

TABLE 1
Inhibition of CIC-1 ion channel using compounds of the disclosure

| Compound investigated | EC₅₀ (μM) |
|---|---|
| A-3 | 7.4 |
| A-4 | 1.7 |
| A-5 | 6.6 |
| A-6 | 5.8 |
| A-7 | 5.3 |
| A-8 | 3.2 |
| A-9 | 4.3 |
| A-10 | 4.1 |
| A-13 | 4.4 |
| A-14 | 3.2 |

### Example 16: Measurement of Force in an In Vitro Model

The current disclosure relates to compounds that inhibit CIC-1 ion channels and increase muscle excitability and thereby improve muscle function in clinical conditions where muscle activation is failing. Such conditions result in loss of contractile function of skeletal muscle, weakness and excessive fatigue. In this series of experiments the compounds were tested for their ability to restore contractile function of isolated rat muscle when the neuromuscular transmission had been compromised akin to neuromuscular disorders.

Experimentally, soleus muscles from 4-5 week old rats were isolated with the motor nerve remaining attached. The nerve-muscle preparations were mounted in experimental setups that enabled electrical stimulation of the motor nerve. Stimulation of the motor nerve led to activation of the muscle fibres and ensuing force production that was recorded. The nerve-muscle preparations were also in these experiments incubated in the standard Krebs Ringer (see example 15) and the solution was heated to 30° C. and continuously equilibrated with a mixture of 95% O₂ and 5% CO₂, PH ˜7.4.

After mounting the nerve-muscle preparation in the experimental setup, the contractile function of the muscle was initially assessed under the control conditions. Sub-maximal concentration of tubocurarine (115 nM), an acetylcholine receptor antagonist, was then added to the experimental bath to impose partial inhibition of the ability of the motor nerve to activate the muscle fibres. The experimental condition mimics the failing neuromuscular transmission in a range of neuromuscular disorders. After addition of tubocurarine the contractile force declined over the next 90 minutes to 10-50% of the control force. The test compound was then added to obtain the required compound concentration in the bath and the contractile force recovered was measured. To quantify the ability of the compound to restore force the percentage of the initial force that was restored was determined after 40 mins of compound exposure and the point increase is reported in Table 2.

TABLE 2
Percentage increase of initial force that was restored

| | Concentration of test | |
|---|---|---|
| Compound investigated | compound (μM) | Point increase (%) |
| A-1 | 50 | 19 |
| A-2 | 100 | 16 |
| A-3 | 50 | 46 |
| A-4 | 10 | 23 |
| A-5 | 50 | 48 |
| A-6 | 10 | 28 |
| A-7 | 50 | 44 |
| A-8 | 10 | 24 |
| A-9 | 50 | 50 |
| A-10 | 10 | 21 |
| A-11 | 50 | 25 |
| A-12 | 50 | 39 |
| A-13 | 10 | 29 |
| A-14 | 50 | 44 |

In conclusion, this example demonstrates that the compounds of the present disclosure are able to increase muscle excitability and thereby improve muscle function in clinical conditions.

### Example 5: Measurement of In Situ Muscle Contractile Characteristics

Isometric hindlimb force was measured in 12-week old female Lewis rats in the presence and absence of compound.

Rats were placed under anaesthesia with isoflurane (2-4%), intubated and subsequently connected to a micro ventilator (Microvent 1, Hallowell EMC, US). Two stimulation electrodes were inserted through the skin to stimulate the sciatic nerve. A small incision was made proximal to the ankle, to expose the Achilles tendon, which was tied by cotton string, and connected to a force transducer (Fort250, World Precision Instruments) with adjustable position (Vernier control). The Achilles tendon was then cut distal to the attached cotton string. The rat was placed on a heated pad, and to prevent movement artefacts from contraction of the ankle dorsiflexors, the foot was fixated by tape on a footplate.

Muscle contractile properties were assessed by applying an electrical current (under supramaximal voltage conditions) to the nerve and recording the force generated by the muscle. The muscle was stretched until maximal force was obtained, when assessed by 2 Hz stimulation. Isometric force was measured every 30 seconds at 12 Hz (Twitch), 10 pulses, and at every 5 minutes at 80 Hz (Tetanic) for 1 second (80 pulses). This stimulation pattern was employed throughout the experiment, expect in few cases where 80 Hz stimulation was replaced by 12 Hz (10 pulses). Neuromuscular transmission was partially inhibited by constant infusion of Cisatracurium (Nimbex, GlaxoSmithKline) at a concentration of 0.1 mg/kg at an adjustable infusion speed, adjusted individually for each animal to obtain a level of inhibition of ca. 50% of the forced generated at 12 Hz stimulation on the 4th pulse. When the level of neuromuscular inhibition was stable, the test article (sodium salt of test article dissolved in PBS) was injected i.v. or p.o. at the chosen concentration. The effect of test article was assessed on its ability to increase force generated from the stimulation pattern applied. The effect was assessed in the ability to increase force per se (tetanic, 80 Hz, stimulation), and the ratio between individual twitch peaks (12 Hz stimulation). The effect was monitored for at least 1 hour after injection of test article. In addition, the time from injection of test article to maximal effect on force (both twitch and tetanic) was noted and the time for the effect to disappear (return to baseline), if possible. When appropriate the infusion of neuromuscular blocking agent was ceased, with the stimulation pattern continued, and the return of force to control levels was monitored. Animals were sacrificed by cervical dislocation while still fully sedated.

The sodium salt of compound A-7 was dosed 2.5 mg/kg i.v. in PBS resulting in an increase in tetanic force of 27%.

The sodium salt of compound A-14 was dosed 10 mg/kg p.o. in PBS resulting in an increase in tetanic force of 45%.

This demonstrates that compounds of the disclosure can restore force to muscles in vivo which have been partially inhibited by a neuromuscular blocker.

1. A compound of Formula (I): 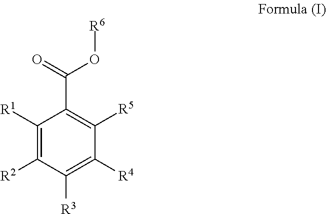
wherein:
R¹ is selected from the group consisting of F; Cl; Br; I; C₁₋₃ alkyl optionally substituted with one or more, identical or different, substituents R⁸; —OC₂₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
R² is selected from the group consisting of C₁₋₃ alkyl optionally substituted with one or more, identical or different, substituents R⁸ and C₂₋₃ alkenyl optionally substituted with one or more, identical or different, substituents R⁸ or when R¹ is —OC₂₋₃ alkyl optionally substituted with one or more, identical or different, substituents R⁷ then R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
R³ is selected from the group consisting of H and F;
R⁴ is selected from the group consisting of F, Cl, Br and I;
R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
R⁷ is independently selected from the group consisting of deuterium, F and Cl;
R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
R⁹ is independently selected from the group consisting of deuterium, methoxy, nitro, cyano, Cl, Br, I, and F;
or a pharmaceutically acceptable salt, hydrate, polymorph, tautomer, or solvate thereof with the proviso that when R¹ is F or Me, R² is Me, R³ is H, R⁴ is Cl or Br and R⁵ is OMe, then R⁶ is not H, Me or Et; and with the proviso that when R¹ is Cl or Br, R² is Me, R³ is H, R⁴ is F and R⁵ is OMe, then R⁶ is not H, Me or Et; and with the proviso that when R¹ is F, R² is Me, R³ is F, R⁴ is F and R⁵ is OMe, then R⁶ is not H.

2. The compound according to claim 1, wherein R¹ is Me.

3. The compound according to claim 1, wherein R² is Me.

4. The compound according to claim 1, wherein R³ is H.

5. The compound according to claim 1, wherein R⁴ is F or Cl.

6. The compound according to claim 1, wherein R⁵ is selected from the group consisting of —OMe, —OCH₂F, —OCHF₂, —OCF₃ and —SMe.

7. The compound according to claim 1, wherein R⁶ is H.

8. The compound according to claim 1, wherein the compound is selected from the group consisting of:
3-chloro-2-cyclopropoxy-5,6-dimethylbenzoic acid;
2,5-dichloro-6-methoxy-3-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid; and
3-chloro-2-ethoxy-5,6-dimethylbenzoic acid.

9. A composition comprising the compound according to claim 1 and a pharmaceutically acceptable carrier.

10. (canceled)

11. A method for treating a disease comprising administering the compound according to claim 1 to a person in need thereof, wherein the disease is selected from the group consisting of myasthenia gravis, autoimmune myasthenia gravis, congenital myasthenic syndrome, seronegative myasthenia gravis, muscle specific kinase myasthenia gravis (MuSK-MG), Lambert-Eaton Syndrome, critical illness myopathy, amyotrophic lateral sclerosis (ALS), spinal muscular atrophy (SMA), critical illness myopathy (CIM), Charcot-Marie Tooth disease, diabetic polyneuropathy, periodic paralysis, hypokalemic periodic paralysis, hyperkalemic periodic paralysis, myotubular myopathy, Duchenne muscular dystrophy, Guillain-Barre syndrome, poliomyelitis, post-polio syndrome, chronic fatigue syndrome, critical illness polyneuropathy, metabolic myopathy, Kennedy's disorder, multiple sclerosis, sarcopenia and multifocal motor neuropathy.

12. The method according to claim 11, wherein the disease is sarcopenia.

13. The method according to claim 11, wherein the administering of the compound comprises reversing and/or ameliorating a neuromuscular blockade.

14. A compound of Formula (I): 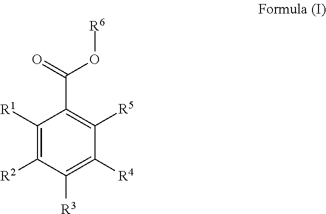
wherein:
R¹ is selected from the group consisting of F; Cl; Br; I; C₁₋₃ alkyl optionally substituted with one or more, identical or different, substituents R⁸; —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
R² is selected from the group consisting of F; Cl; Br; I; C₁₋₃ alkyl optionally substituted with one or more, identical or different, substituents R⁸ and C₂₋₃ alkenyl optionally substituted with one or more, identical or different, substituents R⁸;
R³ is selected from the group consisting of H and F;
R⁴ is selected from the group consisting of F, Cl, Br and I;
R⁵ is selected from the group consisting of —OC₁₋₃ alkyl optionally substituted with one or more, identical or different, substituents R⁷; —OC₃₋₅ cycloalkyl optionally substituted with one or more, identical or different, substituents R⁷; —SC₁₋₃ alkyl optionally substituted with one or more, identical or different, substituents R⁷ and —SC₃₋₅ cycloalkyl optionally substituted with one or more, identical or different, substituents R⁷;
R⁶ is selected from the group consisting of H; C₁₋₅ alkyl optionally substituted with one or more, identical or different, substituents R⁷; C₂₋₅ alkenyl optionally substituted with one or more, identical or different, substituents R⁷; C₂₋₅ alkynyl optionally substituted with one or more, identical or different, substituents R⁷; C₃₋₆ cycloalkyl optionally substituted with one or more, identical or different, substituents R⁷; phenyl optionally substituted with one or more, identical or different, substituents R⁹; and benzyl optionally substituted with one or more, identical or different, substituents R⁹;
R⁷ is independently selected from the group consisting of deuterium, F and Cl;
R⁸ is independently selected from the group consisting of deuterium, F, Cl, —OC₁₋₅ alkyl, —SC₁₋₅ alkyl, —N(═O)—C₁₋₅ alkyl and —N(C₁₋₅ alkyl)-C(═O)C₁₋₅ alkyl; and
R⁹ is independently selected from the group consisting of deuterium, methoxy, nitro, cyano, Cl, Br, I, and F;
or a pharmaceutically acceptable salt, hydrate, polymorph, tautomer, or solvate thereof for use in treating, ameliorating and/or preventing a neuromuscular disorder, and/or for use in reversing and/or ameliorating a neuromuscular blockade.

15. The compound for use according to claim 14, wherein the compound is selected from the group consisting of:
3-bromo-5-fluoro-2,6-dimethoxybenzoic acid;
3-fluoro-2,6-dimethoxy-5-methylbenzoic acid;
5-chloro-2-methyl-3-[(methylsulphanyl)methyl]benzoic acid;
3,5-dichloro-2,6-dimethoxybenzoic acid;
3-chloro-2-cyclopropoxy-5,6-dimethylbenzoic acid;
2,5-dichloro-6-methoxy-3-methylbenzoic acid;
3-chloro-2,6-dimethoxy-5-methylbenzoic acid;
3-chloro-5,6-dimethyl-2-(methylsulphanyl)benzoic acid;
3-chloro-5,6-dimethyl-2-(trifluoromethoxy)benzoic acid;
3-chloro-2-(difluoromethoxy)-5,6-dimethylbenzoic acid;
3-chloro-2-(fluoromethoxy)-5,6-dimethylbenzoic acid;
3-fluoro-2-methoxy-5,6-dimethylbenzoic acid;
3-chloro-2-ethoxy-5,6-dimethylbenzoic acid;
3-bromo-2-methoxy-5,6-dimethylbenzoic acid; and
3-chloro-2-methoxy-5,6-dimethylbenzoic acid.

16. The method of claim 11, wherein the compound is administered as a composition containing the compound and a pharmaceutically acceptable carrier.

17. The method of claim 12, wherein the compound is administered as a composition containing the compound and a pharmaceutically acceptable carrier.

18. The method of claim 13, wherein the compound is administered as a composition containing the compound and a pharmaceutically acceptable carrier.
